(19) United States
(12) Patent Application Publication
(10) Pub. No.: US 2025/0325237 A1
(43) Pub. Date: Oct. 23, 2025

(54) CLINICAL EDUCATION METHOD AND SYSTEM

(71) Applicant: Etiometry Inc., Boston, MA (US)

(72) Inventor: Dimitar V. Baronov, Weston, MA (US)

(21) Appl. No.: 19/186,412

(22) Filed: Apr. 22, 2025

Related U.S. Application Data
(60) Provisional application No. 63/637,039, filed on Apr. 22, 2024.

(51) Int. Cl. A61B 5/00; G16H 10/60

(52) U.S. Cl. CPC A61B 5/7275; A61B 5/7267; G16H 10/60

(57) ABSTRACT

A method and system enhances patient care through a sophisticated data-driven decision support platform. Initially, it begins by receiving patient data, which lays the groundwork for the method. Then, it proceeds to use this patient data to ascertain one or more possible medical conditions that the patient might have. In response to these conditions, the method involves guiding a user—likely a healthcare practitioner—to appropriate resources that are specifically chosen based on the identified conditions, favorably facilitating further medical understanding, treatment, or investigation.

### RELATED APPLICATIONS

This application claims priority to U.S. Provisional Application No. 63,637,039, filed Apr. 22, 2024 and titled “Clinical Education M ethod and System” and naming DimitarV. Baronov as inventor [Attorney Docket No. 3816-12701].

This application is related to the following patents:
U.S. Pat. No. 10,062,456;
U.S. Pat. No. 10,796,804;
U.S. Pat. No. 11,557,394;
U.S. Pat. No. 11,676,730;
U.S. Pat. No. 11,763,947;
U.S. Pat. No. 11,482,336;
U.S. Pat. No. 11,183,467;
U.S. Pat. No. 12,272,462.

The disclosure of each of the foregoing is incorporated herein, in its entirety, by reference.

### GOVERNMENT LICENSE RIGHTS

This invention was made with government support under R43HL117340 awarded by the National Heart, Lung, And Blood Institute of the National Institutes of Health. The government has certain rights in the invention.

### TECHNICAL FIELD

Illustrative embodiments of the invention generally relate to medical technology and, more particularly, various embodiments of the invention relate to an educational platform to assist in clinical diagnosis and treatment of patients. Background A rt

In the realm of healthcare analytics, there is a targeted focus on detecting and diagnosing specific patient conditions. One aim of such analytics is to leverage data to facilitate early diagnosis and tailor patient care more effectively. By sifting through large volumes of health data and identifying patterns, these tools can provide critical insights that might not be apparent to healthcare providers at first glance. Undesirably, the appropriate resources often are not readily apparent to the clinician.

### SUMMARY OF VARIOUS EMBODIMENTS

In accordance with an illustrative embodiment, a computer-implemented method includes:
receiving, at a computer system, patient data describing patient physiology, said patient data including at least data quantifying a set of patent state variables from a set of sensors coupled to the patient;
producing, by the computer system from the patient data, a set of patient parameters for the patient, said patient parameters including: (1) a probability that the patient is in a specific patient state;
tokenizing, by the computer system, the patient parameters to produce a set of text descriptions; and
producing, by the computer system, from the set of text descriptions, a structured text prompt configured as input to a large language model, said structured text prompt configured to cause the large language model to generate at least one of: (a) a set of proposed diagnoses of the patient, each proposed diagnosis including a quantitative probabilistic assessment; (b) a physiological interpretation of patterns observed within the patient parameters; (c) a listing of relevant clinical considerations; (d) a listing of relevant clinical caveats to the set of proposed diagnoses; (e) a suggestion of an additional assessment to be made for the patient; (f) a suggestion for additional monitoring of the patient; (g) a listing of clinical guidelines relevant to a specific patient state or diagnosis of the patient; (h) a listing of literature relevant to a specific patient state or diagnosis of the patient; and (i) an indication whether the patient is compliant with a particular protocol.

In some embodiments, producing, from the patient data, a set of patient parameters for the patient includes producing (2) a quantitative indicator of patient eligibility for a specified treatment protocol.

In some embodiments, the structured text prompt configured to cause the large language model to generate (j) an indication whether the patient is compliant with a specified treatment protocol.

In some embodiments, tokenizing, by the computer system, the patient parameters to produce a set of text descriptions includes: correlating the patient parameters to a pre-existing form text string; and modifying the pre-existing form text string to add the patient parameters.

In some embodiments, tokenizing, by the computer system, the patient parameters to produce a set of text descriptions includes: providing the patient parameters to a neural network trained to recognize a specific pattern within patient parameters; and receiving an output from the neural network which output correlates the patient parameters to a pre-existing form text string which pre-existing form text string correlated to the specific pattern; and modifying the pre-existing form text string to add the patient parameters.

In some embodiments, the patient parameters include a plurality of patient parameters spanning a length of time; and the set of text descriptions includes text describing a trend over the length of time, the trend represented by the plurality of patient parameters spanning a length of time. In some embodiments, the patient parameters define an evolution of the patient state over time.

In some embodiments, the set of text descriptions include a description of the patient's eligibility for the specified treatment protocol.

In some embodiments, the set of text descriptions include a description of the patient's concordance with the specified treatment protocol.

In some embodiments, the set of text descriptions includes a request to identify literature relating to a specific patient state.

Another embodiment includes a computer-implemented system including: a communications interface configured to receive patient data describing patient physiology, said patient data including at least data quantifying a set of patent state variables from a set of sensors coupled to the patient; a pattern recognition engine configured to produce, from the patient data, a set of patient parameters for the patient, said patient parameters including: (1) a probability that the patient is in a specific patient state; a tokenizing engine configured to produce, from the patient parameters, a set of text descriptions; and a prompt generator configured to produce, from the set of text descriptions, a structured text prompt configured as input to a large language model, said structured text prompt configured to cause the large language model to generate at least one of: (a) a set of proposed diagnoses of the patient, each proposed diagnosis including a quantitative probabilistic assessment; (b) a physiological interpretation of patterns observed within the patient parameters; (c) a listing of relevant clinical considerations; (d) a listing of relevant clinical caveats to the set of proposed diagnoses; (e) a suggestion of an additional assessment to be made for the patient; (f) a suggestion for additional monitoring of the patient; (g) a listing of clinical guidelines relevant to a specific patient state or diagnosis of the patient; (h) a listing of literature relevant to a specific patient state or diagnosis of the patient; and/or (i) an indication whether the patient is compliant with a particular protocol.

In some embodiments, the patient parameters further include: (2) a quantitative indicator of patient eligibility for a specified treatment protocol.

In some embodiments, the structured text prompt configured to cause the large language model to generate (j) an indication whether the patient is compliant with a specified protocol.

In some embodiments, the tokenizing engine is configured to produce the set of text descriptions by: correlating the patient parameters to a pre-existing form text string; and modifying the pre-existing form text string to add the patient parameters.

Some embodiments further include: a neural network trained to recognize a specific pattern within patient parameters; and wherein the tokenizing engine is configured to: receive an output from the neural network which output correlates the patient parameters to a pre-existing form text string which pre-existing form text string correlated to the specific pattern; and modify the pre-existing form text string to add the patient parameters.

Another embodiment includes a non-transitory computer-readable medium having computer executable code thereon, the computer executable code, when executed by a computer system, causing the computer system to perform a method, the code including: code for receiving, at the computer system, patient data describing patient physiology, said patient data including at least data quantifying a set of patent state variables from a set of sensors coupled to the patient; code for producing, by the computer system from the patient data, a set of patient parameters for the patient, said patient parameters including (1) a probability that the patient is in a specific patient state; code for tokenizing, by the computer system, the patient parameters to produce a set of text descriptions; and code for producing, by the computer system, from the set of text descriptions, a structured text prompt configured as input to a large language model, said structured text prompt configured to cause the large language model to generate at least one of: (a) a set of proposed diagnoses of the patient, each proposed diagnosis including a quantitative probabilistic assessment; (b) a physiological interpretation of patterns observed within the patient parameters; (c) a listing of relevant clinical considerations; (d) a listing of relevant clinical caveats to the set of proposed diagnoses; (e) a suggestion of an additional assessment to be made for the patient; (f) a suggestion for additional monitoring of the patient; (g) a listing of clinical guidelines relevant to a specific patient state or diagnosis of the patient; (h) a listing of literature relevant to a specific patient state or diagnosis of the patient; and (i) an indication whether the patient is compliant with a particular protocol.

In some embodiments, producing, from the patient data, a set of patient parameters for the patient includes producing (2) a quantitative indicator of patient eligibility for a specified treatment protocol.

In some embodiments, tokenizing the patient parameters to produce a set of text descriptions includes: correlating the patient parameters to a pre-existing form text string; and modifying the pre-existing form text string to add the patient parameters.

In some embodiments, tokenizing the patient parameters to produce a set of text descriptions includes: providing the patient parameters to a neural network trained to recognize a specific pattern within patient parameters; and receiving an output from the neural network which output correlates the patient parameters to a pre-existing form text string which pre-existing form text string correlated to the specific pattern; and modifying the pre-existing form text string to add the patient parameters.

In some embodiments, the patient parameters include a plurality of patient parameters spanning a length of time; the set of text descriptions includes text describing a trend over the length of time, the trend represented by the plurality of patient parameters spanning a length of time.

In accordance with one embodiment of the invention, a method enhances patient care through a sophisticated data-driven decision support platform. Initially, it begins by receiving patient data, which lays the groundwork for the method. Then, it proceeds to use this patient data to ascertain one or more possible medical conditions that the patient might have. In response to these conditions, the method involves guiding a user—likely a healthcare practitioner—to appropriate resources that are specifically chosen based on the identified conditions, favorably facilitating further medical understanding, treatment, or investigation.

The patient data could encompass a wide array of information, including both current clinical information, which pertains to recent observations or reports about the patient, and stored clinical information from previous encounters in the patient's medical history. It can also be extensive, including details such as age, gender, demographics, treatment history, family medical history, medications currently being taken, known allergies, diagnostic test results, and immunization statuses.

Among other things, the “resource” may include a database with clinical information that can be used for reference. The method is furthered by the ability to display this resource on the graphical user interface of a display device, enhancing the ability for users to interact with and visualize data conveniently. Additionally, the method contemplates a user may be directed by navigating them to access the resource through the Internet, which suggests the incorporation of online tools and resources that can be remotely accessed.

There is also the feature of a graphical user interface displaying clinical information about the patient, which can have elements that allow the user to select and interact with the resource highlighted by the system. Despite the comprehensive nature of the method in facilitating condition identification and resource direction, it is expressly noted that the method preferably stops short of delivering a clinical diagnosis. This delineates the method's utility as a support tool that assists in decision-making that does not replace professional medical diagnosis.

In essence, various embodiments integrate the analysis of patient data with strategic resource direction, all delivered through an intuitive digital interface, thereby optimizing the information available to healthcare providers and potentially improving patient outcomes.

Illustrative embodiments of the invention are implemented as a computer program product having a computer usable medium with computer readable program code thereon. The computer readable code may be read and utilized by a computer system in accordance with conventional processes.

### BRIEF DESCRIPTION OF THE DRAWINGS

The foregoing features of embodiments will be more readily understood by reference to the following detailed description, taken with reference to the accompanying drawings, in which:

FIG. 1A and FIG. 1B schematically illustrate an embodiment of a medical care risk-based monitoring environment in accordance with the disclosure;

FIG. 2A illustrates conceptually a basic schematic of the physiology observer module in accordance with the disclosure;

FIG. 2B schematically illustrates an embodiment of a predict module;

FIG. 2C schematically illustrates an embodiment of an update module;

FIG. 2D, FIG. 2E and FIG. 2F illustrate conceptually exemplary graphs of probability density functions for select ISVs as generated by the physiology observer module in accordance with the disclosure;

FIG. 3 illustrates conceptually a non-limiting example of a physiology observer process in accordance with the disclosure;

FIG. 4A illustrates conceptually a non-limiting example of the physiology observer process in accordance with the disclosure;

FIG. 4B illustrates a method applying intermittent laboratory data through the physiology observer module to achieve better accuracy in an estimated ISV PDF;

FIG. 5 illustrates conceptually a time line, wherein back propagation is used to incorporate information in accordance with the disclosure;

FIG. 6 illustrates conceptually an example of a process involving mean arterial blood pressure (ABPm) in accordance with the disclosure;

FIG. 7 illustrates conceptually an example of resampling in accordance with the disclosure;

FIG. 8A and FIG. 8B each schematically illustrates, respectively, an embodiment of a clinical trajectory interpreter module using joined Probability Density Functions of ISVs and performing state probability estimation to calculate the probabilities of different patient states in accordance with the disclosure;

FIG. 8C, FIG. 8D, FIG. 8E, FIG. 8F and FIG. 8G each schematically illustrates, respectively, a method of determining a patient stage using probability density functions of internal state variables.

FIG. 9 illustrates conceptually a non-limiting example of a definition of a patient state employed by the clinical trajectory interpreter module in accordance with the disclosure;

FIG. 10 illustrates conceptually a non-limiting example of how a clinical trajectory interpreter module may employ the definition of patient states to assign probabilities that the patient may be classified under each of the four possible patient states at a particular point of time;

FIG. 11 illustrates conceptually an alternative approach of estimating the probabilities for different patient states in accordance with the disclosure;

FIG. 12 illustrates conceptually a non-limiting example of a definition of patient states assigned with hazard levels by the clinical trajectory interpreter module in accordance with the disclosure;

FIG. 13 illustrates conceptually patient states and their respective probabilities organized into tree graphs called etiologies in accordance with the disclosure;

FIG. 14 illustrates conceptually an exemplary etiology tree for a given set of patient states and physiologic variables in accordance with the disclosure;

FIG. 15 schematically illustrates an embodiment of a system;

FIG. 16A is a flowchart of an embodiment of a method for determining whether a patient is eligible for a clinical protocol;

FIG. 16B schematically illustrates an embodiment of a clinical protocol;

FIG. 17 is a flowchart of an embodiment of a method;

FIG. 18 schematically illustrates protocol criteria;

FIG. 19A schematically shows a knowledge platform in accordance with illustrative embodiments.

FIG. 19B schematically illustrates an embodiment of a system flow;

FIG. 20 shows a process the knowledge platform may use to direct users to resources for diagnosing and/or treating a patient in accordance with illustrative embodiments.

FIG. 21 schematically shows an example of a graphical user interface implementing a knowledge platform in accordance with illustrative embodiments.

FIG. 22 schematically shows details of a system in accordance with illustrative embodiments.

FIG. 23 is a flowchart of an embodiment of a method;

FIG. 24 schematically illustrates a system.

### DETAILED DESCRIPTION OF ILLUSTRATIVE EMBODIMENTS

In illustrative embodiments, using patient information, a knowledge platform directs clinicians toward educational resources that can help diagnose and/or treat the patient. To that end, the knowledge platform may use past patient information, such as allergy information, test data, etc., with current clinical and non-clincical patient information to formulate a potential set of conditions. Then, using that condition, the knowledge platform may direct users toward resources to diagnose and treat the condition. Details of illustrative embodiments are discussed below.

Definitions: As used in this description and the accompanying claims, the following terms shall have the meanings indicated, unless the context otherwise requires.

The term “clinical risk” means the probability of a patient being in a particular patient state, for example at a particular time.

The term “clinical trajectory” means the sequence of patient states through which a patient evolves during a patient's clinical course.

The term “hidden,” in reference to an internal state variable, means an ISV that is not directly measured by a sensor coupled to the patient. Some hidden ISVs cannot be directly measured by a sensor coupled to the patient. Some hidden ISVs require laboratory analysis of a sample (e.g., blood) taken from the patient. As described below, some hidden ISVs may be generated from measurements of ISVs that are not hidden, and may be referred-to as “generated internal state variables.”

The term “internal state variable” (or “ISV”) means a parameter of a patient's physiology that is physiologically relevant to one of a treatment and a condition of a patient.

Examples of ISVs include, without limitation, ISVs that are directly observable with noise (as a non-limiting example, heart rate is a directly observable ISV), ISVs that are hidden (as a non-limiting example, alveolar dead space, oxygen delivery (DO2) defined as the flow of blood saturated oxygen through the aorta cannot be directly measured and is thus hidden), or measured intermittently (as a non-limiting example, hemoglobin concentration as measured from Complete Blood Count tests is an intermittently observable ISV). Other examples of ISVs include, without limitation, Pulmonary Vascular Resistance (PVR); Cardiac Output (CO); hemoglobin, and rate of hemoglobin production/loss.

The term “nominal” in reference to a datum for a patient means a value that is nominal for a population to which the patient belongs. For example, a patient to which the patient belongs may be defined as a population of patients of the same age, and/or a population of patients of the same gender.

The term “null” in reference to a datum for a patient means an empty measurement value. Substituting a null value for the value of an as-measured datum simulates a scenario in which the as-measured datum was not received by the system.

The term “patient state” means a qualitative description of the physiology of a patient at a particular point of time of the patient's clinical course, which qualitative description is derived from quantified evidence (e.g., measurements of one or more of the patient's internal state variables), and which qualitative description is recognizable by medical practice, and may have implications to clinical decision-making. A patient state may be a medical condition, such as an adverse medical condition, for example. The term “patient state” does not include the patient's state of consciousness (e.g., awake and/or asleep; comatose; conscious; in the process of waking up; in the process of falling asleep; etc.)

Examples of particular patient states include, but are not limited to, adverse medical conditions such as inadequate delivery of oxygen, inadequate ventilation of carbon dioxide, hyperlactatemia, acidosis; cardiogenic shock; amongst others. In addition, these patient states may be specific to a particular medical condition, and the bounds of each of the patient states may be defined by threshold values of various physiological variables and data.

A “set” includes at least one member. Unless otherwise specified, a set may include as few as a single member, or may include a plurality of members.

### System Modules and Interaction

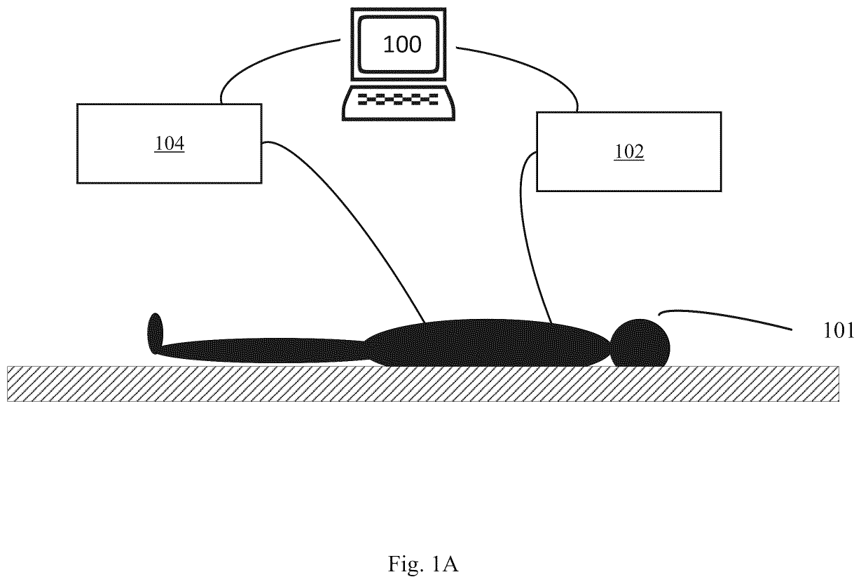

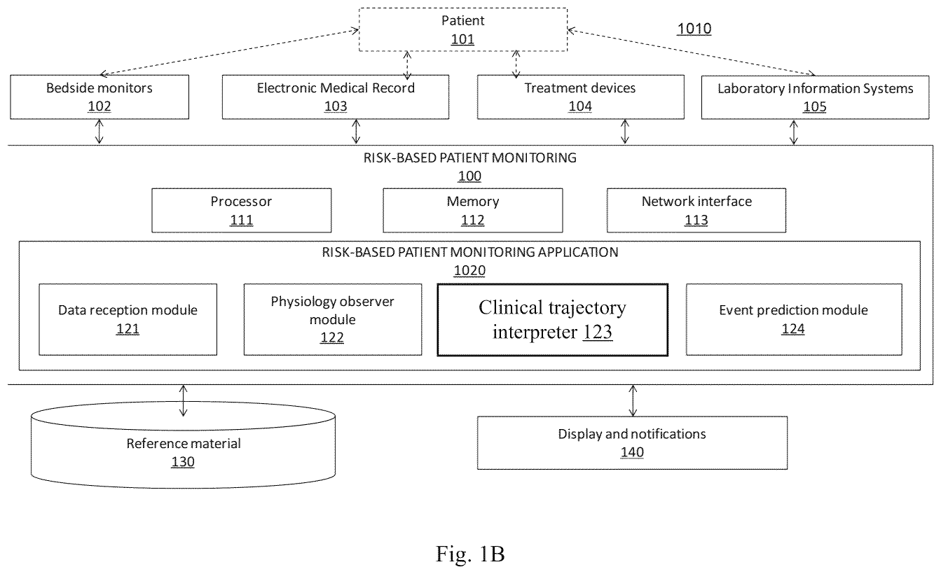

Referring now to the figures, FIG. 1A and FIG. 1B illustrate an embodiment of a medical care risk-based monitoring environment 1010 for providing health providers, such as physicians, nurses, or other medical care providers, risk-based monitoring in accordance with various embodiments of the present disclosure. A patient 101 may be coupled to one or more physiological sensors or bedside monitors 102 that may monitor various physiological parameters of the patient. It should be noted that a patient may be a human, or not human (a non-human being).

These physiological sensors may include but are not limited to, a blood oximeter, a blood pressure measurement device, a pulse measurement device, a glucose measuring device, one or more analyte measuring devices, an electrocardiogram recording device, amongst others. In addition, the patient may be administered routine exams and tests and the data stored in an electronic medical record (EMR) 103. The electronic medical record 103 may include but is not limited to stored information such as hemoglobin, arterial and venous oxygen content, lactic acid, weight, age, sex, ICD-9 code, capillary refill time, subjective clinician observations, patient self-evaluations, prescribed medications, medications regiments, genetics, etc. In addition, the patient 101 may be coupled to one or more treatment devices 104 that are configured to administer treatments to the patient. In some embodiments, one or more treatment devices 104 may be controlled by a system 100 as disclosed herein, for example in response to output defining a patient state or medical condition from a trajectory interpreter module. In various embodiments, the treatments devices 104 may include extracorporeal membrane oxygenator, ventilator, medication infusion pumps, etc.

By way of the present disclosure, the patient 101 may be afforded improved risk-based monitoring over existing methods. A patient specific risk-based monitoring system, generally referred to herein as system 100, may be configured to receive patient related information, including real-time information from bed-side monitors 102, EMR patient information from electronic medical record 103, information from treatment devices 104, such as settings, infusion rates, types of medications, and other patient related information, which may include the patient's medical history, previous treatment plans, results from previous and present lab work, allergy information, predispositions to various conditions, and any other information that may be deemed relevant to make an informed assessment of the possible patient conditions and states, and their associated probabilities. For the sake of simplicity, the various types of information listed above will generally be referred to hereinafter as “patient-specific information”. In addition, the system may be configured to utilize the received information, determine the clinical risks, which then can be presented to a medical care provider, including but not limited to a physician, nurse, or other type of clinician.

The system, in various embodiments, includes one or more of the following: a processor 111, a memory 112 coupled to the processor 111, and a network interface 113 configured to enable the system to communicate with other devices over a network. In addition, the system may include a risk-based monitoring application 1020 that may include computer-executable instructions, which when executed by the processor 111, cause the system to be able to afford risk based monitoring of the patients, such as the patient 101.

The risk based monitoring application 1020 includes, for example, a data reception module 121, a physiology observer module 122, a clinical trajectory interpreter module 123 (or, in some embodiments, risk calculation engine 123), and a visualization and user interaction module 124. In an exemplary embodiment, the data reception module 121 may be configured to receive data from bedside monitors 102, electronic medical records 103, treatment devices 104, and any other information that may be deemed relevant to make an informed assessment regarding the patient's clinical risks, and any combination thereof of the preceding elements.

The physiology observer module 122 utilizes multiple measurements to estimate probability density functions (PDF) of internal state variables (ISVs) that describe the components of the physiology relevant to the patient treatment and condition in accordance with a predefined physiology model. The ISVs may be directly observable with noise (as a non-limiting example, heart rate is a directly observable ISV), hidden (as a non-limiting example, oxygen delivery (DO₂) defined as the flow of blood saturated oxygen through the aorta cannot be directly measured and is thus hidden), or measured intermittently (as a non-limiting example, hemoglobin concentration as measured from Complete Blood Count tests is an intermittently observable ISV). In some embodiments, when the physiology observer module 122 evaluates a set of ISVs at a given time step (e.g., tk; tₖ₊₁; generally tₖ₊ₙ), the system 100 may not have a complete set of ISV measurements contemporaneous with that given time step. For example, the system 100 may have measurements for that given time step for some internal state variables, but may not have measurements for that given time step for some other internal state variables (e.g., a contemporaneous measurement for an intermittent ISV may not be available for the given time step). Consequently, that intermittent ISV is, for purposes of evaluating ISVs at the given time step, a hidden ISV. However, evaluation of the set of ISVs by the physiology observer module 122 (as described herein) is nevertheless possible according to embodiments described herein because the predicted PDFs of ISVs 211 carry in them the influence of past measurements of that intermittent ISV, and consequently those predicted PDFs of ISVs 211 are, in illustrative embodiments, sufficient input for the physiology observer module 122.

In one embodiment, instead of assuming that all variables can be estimated deterministically without error, the physiology observer module 122 of the present disclosure provides probability density functions as an output. Additional details related to the physiology observer module 122 are provided herein.

The clinical trajectory interpreter module 123 may be configured, for example, with multiple possible patient states, and may determine which of those patient states are probable and with what probability, given the estimated probability density functions of the internal state variables. Examples of particular patient states include, but are not limited to, hypotension with sinus tachycardia, hypoxia with myocardial depression, compensated circulatory shock, cardiac arrest, hemorrhage, amongst others. In addition, these patient states may be specific to a particular medical condition, and the bounds of each of the patient states may be defined by threshold values of various physiological variables and data. In various embodiments, the clinical trajectory interpreter module 123 may determine the patient conditions under which a patient may be categorized using any of information gathered from reference materials, information provided by health care providers, other sources of information. The reference materials may be stored in a database or other storage device 130 that is accessible to the risk based monitoring application 1020 via network interface 113, for example. These reference materials may include material synthesized from reference books, medical literature, surveys of experts, physician provided information, and any other material that may be used as a reference for providing medical care to patients. In some embodiments, the clinical trajectory interpreter module 123 may first identify a patient population that is similar to the subject patient being monitored. By doing so, the clinical trajectory interpreter module 123 may be able to use relevant historical data based on the identified patient population to help determine the possible patient states.

The clinical trajectory interpreter module 123 is capable of also determining the probable patient states under which the patient can be currently categorized, given the estimated probability density functions of the internal state variables, as provided by physiology observer module 122. In this way, each of the possible patient states is assigned a probability value from 0 to 1. The combination of patient states and their probabilities is defined as the clinical risk to the patient. Additional details related to the clinical trajectory interpreter module 123 are provided herein.

Visualization and user interactions module 124 may be equipped to take the outputs of the data reception module 121 the physiology observer module 122, and the clinical trajectory interpreter module 123 and present them to the clinical personnel. The visualization and user interactions module 124 may show the current patient risks, their evolution through time, the probability density functions of the internal state variables as functions of time, and other features that are calculated by the two modules 122 and 123 as by-products and are informative to medical practice. Additionally, visualization and user interactions module 124 enables the users to set alarms based on the patient state probabilities, share those alarms with other users, take notes related to the patient risks and share those notes with other users, and browse other elements of the patient medical history. Additional details related to the visualization and user interactions module 124 are provided herein.

### I. Physiology Observer Module 122

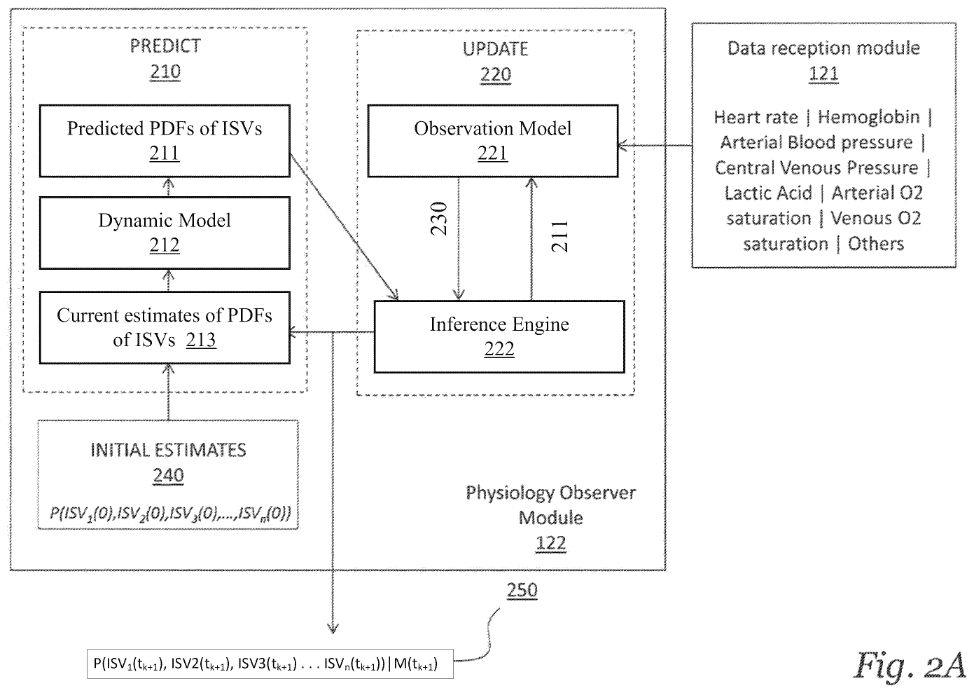

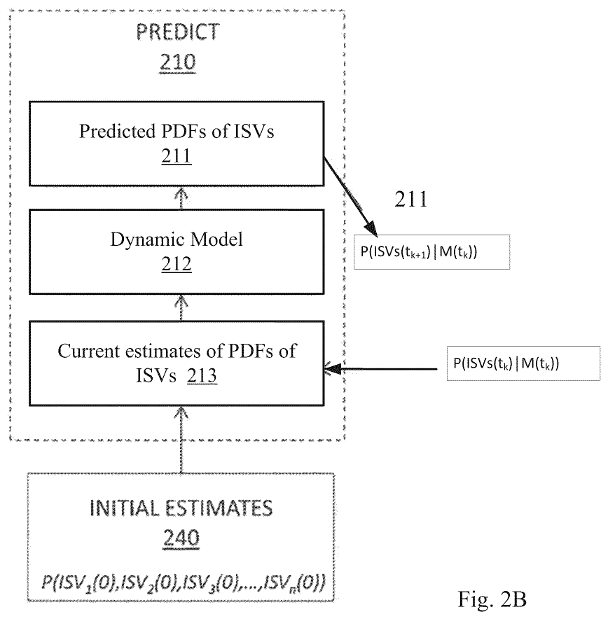

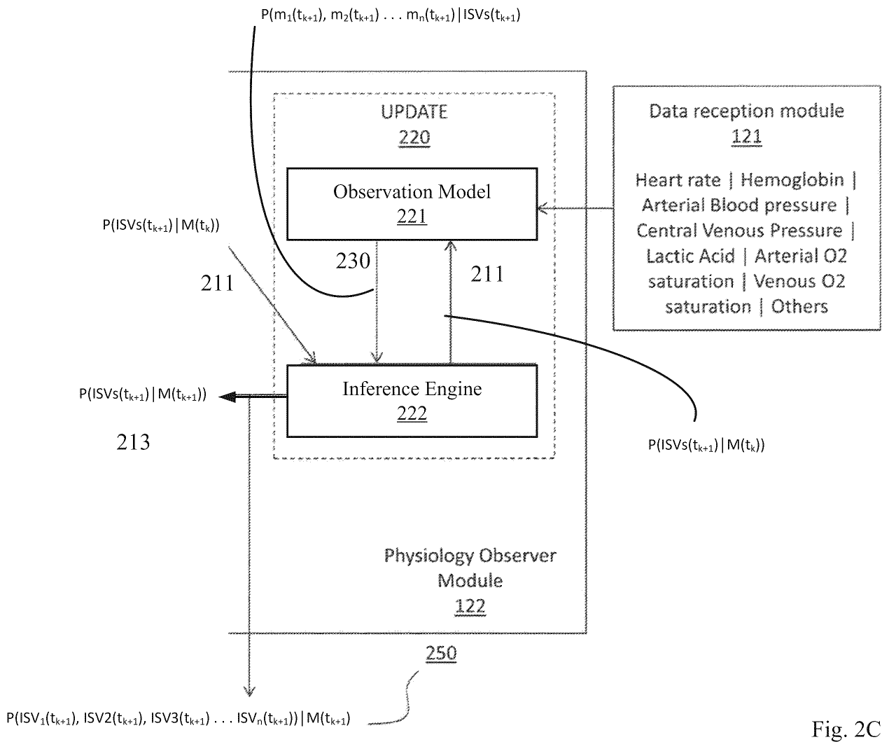

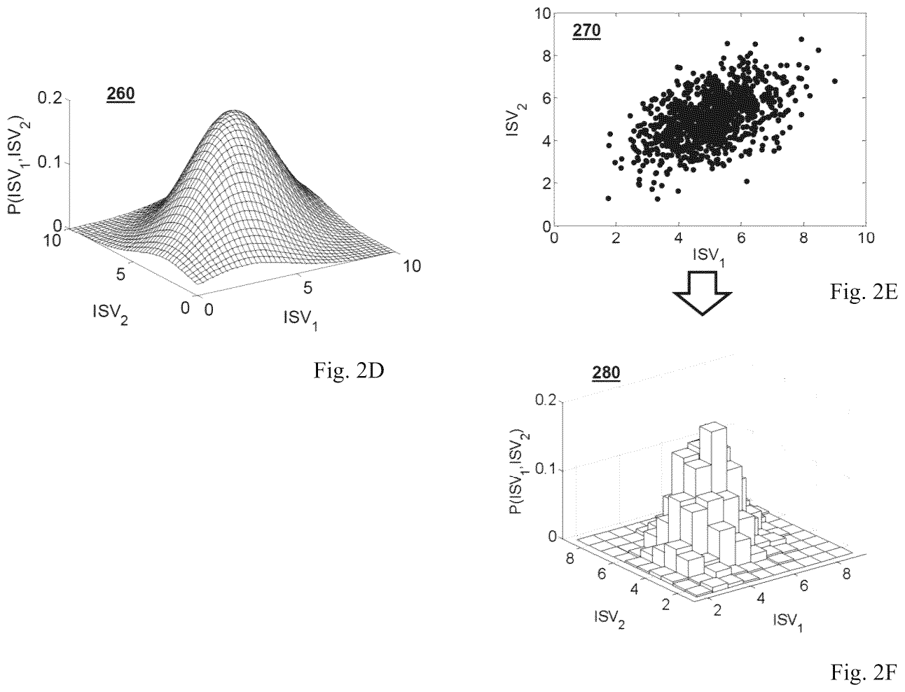

FIG. 2A illustrates a basic schematic of the physiology observer module 122, which utilizes two models of the patient physiology: a dynamic model (or dynamic module) 212 and an observation model (or observation module) 221. The dynamic model 212 captures the relationship arising between the internal state variables at some time tₖ and another close, subsequent time tₖ₊₁, thereby enabling modeling of the patient physiology as a system whose present state has information about the possible future evolutions of the system. Given the propensity of the patient physiology to remain at homeostasis through auto-regulation, and the physical laws guiding different processes in the human body, e.g. fluid mechanics, chemical reactions; there is a clear rational of introducing dynamic equations that capture the evolution of the system from a present state to a future state.

The observation model 221 may capture the relationships between measured physiology variables and other internal state variables. Examples of such models include: a) the dependence of the difference between systolic and diastolic arterial blood pressures (also called pulse pressure) on the stroke volume; b) the relationship between measured heart rate and actual heart rate; c) the relationship between pulse oximetry and arterial oxygen saturation; and d) any other dependence between measurable and therefore observable parameters and internal state variables.

The physiology observer module 122 functions as a recursive filter by employing information from previous measurements to generate predictions of the internal state variables and the likelihood of probable subsequent measurements (i.e., future measurements, relative to the previous measurements) and then comparing them with subsequent measurements (e.g., the most recently acquired measurements). Specifically the physiology observer module 122 utilizes the dynamic model 212 in the predict step or mode 210 and the observation model 221 in the update step or mode 220. In the following illustrative example, operation of the physiology observer module 122 over successive time steps is described using FIG. 2B and FIG. 2C. For purposes of illustration in those figures, the previous time step will be denoted tₖ, the subsequent time step will be denoted tₖ₊₁. It should be noted that the previous time step tₖ, itself was preceded by an earlier time step tₖ₋₁. Consequently, the time steps in this illustrative embodiment proceed from tₖ₋₁ to tₖ to tₖ₊₁.

### A. Predict Module 210 {FIG. 2B}

During the prediction mode 210, at or after time step tₖ and on or before time step tₖ₊₁, the physiology observer module 122 takes the estimated probability density functions (PDFs) of ISVs 213 at a time step tₖ (which were produced at time step tₖ based in part from data from earlier time step tₖ₋₁, and which may be referred-to as the posterior probabilities for time step tₖ) and feeds them to the dynamic model 212, which produces predictions of the probability density functions of the ISVs 211 for the next time step tₖ₊₁.

This is accomplished using the following equation:

$$P ( ISVs ( {t}_{k + 1} ) \vert M ( {t}_{k} ) ) = {\int }_{ISVs ∈ ISV} P ( ISVs ( {t}_{k + 1} ) \vert ISVs ( {t}_{k} ) ) P ( ISVs ( {t}_{k} ) \vert M ( {t}_{k} ) ) dISVs$$

$$Where :$$

$$ISVs ( {t}_{k} ) = \{ {ISV}_{1} ( {t}_{k} ) , {ISV}_{2} ( t k ) , {ISV}_{3} ( {t}_{k} ) , … {ISV}_{n} ( {t}_{k} ) \} ;$$

and

M(tₖ) is the set of all measurements up to time tₖ.

The probability P(ISVs(tₖ+1)|ISVs(tₖ)) defines a transition probability kernel describing the dynamic model 212, which defines how the estimated PDFs evolve with time.

The probabilities P(ISVs(tₖ)|M(tₖ)) are provided by the inference engine 222 and arethe posterior probabilities of the ISVs given the measurements acquired at the earlier time step tₖ₋₁.

### B. Update Module 220

During the update mode 220 of the physiology observer module 122, the predicted probability density functions of the ISVs 211 (i.e., which were produced using the predict module and the density function at the preceding time step tₖ) are compared against the measurements received (at time tₖ₊₁) from data reception module 121 with the help of the observation model 221, and as a result the ISVs are updated to reflect the new available information. Processes of the Observation Model 221 are described in more detail, below.

### 1. Observation Model {221}

The observation model 221 consequently produces a conditional likelihood kernel [for example: [P(m₁(tₖ₊₁), m₂ (tₖ₊₁), . . . mₙ(tₖ₊₁)|ISVs(tₖ₊₁))] and provides the conditional likelihood kernel 230 to the Inference Engine 222. The conditional likelihood kernel 230 provided by the observation model 221 determines how likely the currently received measurements are given the currently predicted ISVs. Criteria for determining how likely the currently received measurements are, given the currently predicted ISVs, may be established by the discretion of the system's designer, based on the particular application faced by the designer.

Note that the observation model 221 has two inputs and one output. The inputs are (1) the received measurements from data reception module 121, and (2) the predicted probability density functions of the ISVs 211 [e.g., P(ISVs(tk+1)|M(tk))], provided via the Inference Engine 222, represented by the arrow pointing from the Inference Engine 222 to the Observation Module 221. The output of the observation model 221 is the conditional likelihood kernel 230.

### 2. Operation of the Observation Model 221

The processes of the Observation Model 221 are described below.

Note that the measurements received at the Data Reception Module 121 are individual measurements at discrete points in time, but subsequent processing steps (e.g. Bayes Theorem at the Inference Engine 222, and the operation of the Clinical Trajectory Interpreter Module 123) require PDFs (probability density functions) as inputs, rather than discrete data points. Consequently, one of the functions of the Observation Model 221 is to intake the discrete measurements and output PDFs. This functionality is explained below, along with an (optional) step that reduces noise in the signals.

### A. Comparing the Received Measurements to the Predicted PDF s of ISVs 211 {and Reducing Noise}

During the update mode 210 of the physiology observer module 122, the predicted PDFs of ISVs 211 (which were generated using the predict module and the PDFs of the ISVs at from time step tₖ) are compared against the measurements received at the subsequent time step (tₖ₊₁) from data reception module 121 with the help of the observation model 221, and as a result the ISVs are updated to reflect the new available information.

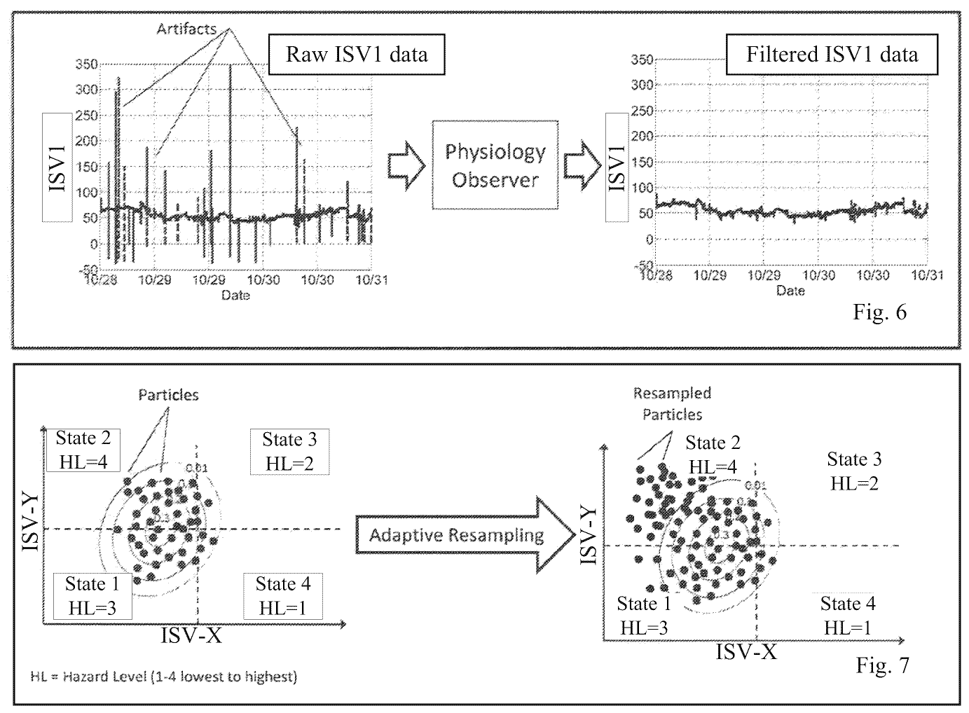

In addition, because physiology observer module 122 maintains estimates of each of the measurements available to the system 100 based on physiologic and statistical models, module 122 may filter artifacts of the measurements that are unrelated to the actual information contained in the measurements. This is performed by comparing the newly acquired measurements with the predicted likelihoods of probable measurements given the previous measurements. If the new measurements are considered highly unlikely by the model, they are not incorporated in the estimation. The process of comparing the measurements with their predicted likelihoods effectively filters artifacts and reduces noise. FIG. 6 shows an example of such a process involving mean arterial blood pressure (ABPm). FIG. 6 shows the raw ABPm measurements prior to being processed by the physiology observer with the measurement artifacts identified, as well as the filtered measurements after being processed by the physiology observer module 122. As can be seen, the measurement artifacts have been removed and the true signal is left.

### B. Creating the Conditional Likelihood Kernel

The “Conditional Likelihood Kernel” 230 [P(m₁(tₖ₊₁), m₂(tₖ₊₁), . . . mₙ(tₖ₊₁)|ISVs(tₖ₊₁))] determines how likely the currently received measurements are given the currently predicted ISVs. A s can be seen from the foregoing formula, the Conditional Likelihood Kernel 230 includes a set of probability density functions of the measurements {mₙ at time tₖ₊₁}assuming the ISVs predicted for time step tₖ₊₁. (i.e, the predicted PDFs of ISVs 211 for time step tₖ₊₁.). Note that from this point forward, the algorithms no longer operate on the discrete measurements from the data reception module per se

In general, creating probably density functions of ISVs (i.e., the components of the Conditional Likelihood Kernel 230) is performed by an “inference scheme.” There are several such inference schemes, including for example exact inference schemes.

In various embodiments, physiology observer module 122 may utilize a number of algorithms for estimation or inference. Depending on the physiology model used, the physiology observer module 122 may use exact inference schemes, such as the Junction Tree algorithm, or approximate inference schemes using Monte Carlo sampling such as a particle filter, or a Gaussian approximation algorithms such as a Kalman Filter or any of its variants.

As discussed, the physiology model used by physiology observer module 122 may be implemented using a probabilistic framework known as a Dynamic Bayesian Network, which graphically captures the causal and probabilistic relationship between the ISVs of the system, both at a single instance of time and over time. Because of the flexibility this type of model representation affords, the physiology observer module 122 may utilize a number of different inference algorithms. The choice of algorithm is dependent on the specifics of the physiology model used, the accuracy of the inference required by the application, and the computational resources available to the system. U sed in this case, accuracy refers to whether or not an exact or approximate inference scheme is used. If the physiology observer model is of limited complexity, then an exact inference algorithm may be feasible to use. In other cases, for more complex physiology observer models, no closed form inference solution exists, or if one does exist, it is not computationally tractable given the available resources. In this case, an approximate inference scheme may be used.

The simplest case in which exact inference may be used, is when all of the ISVs in the physiology model are continuous variables, and relationships between the ISVs in the model are restricted to linear Gaussian relationships. In this case, a standard Kalman Filter algorithm can be used to perform the inference. With such algorithm, the probability density function over the ISVs is a multivariate Gaussian distribution and is represented with a mean and covariance matrix.

When all of the ISV's in the model are discrete variables, and the structure of the graph is restricted to a chain or tree, the physiology observer module 122 may use either a Forward-backward algorithm, or a Belief Propagation algorithm for inference, respectively. The Junction Tree algorithm is a generalization of these two algorithms that can be used regardless of the underlying graph structure, and so the physiology observer module 122 may also use this algorithm for inference. Junction Tree algorithm comes with additional computational costs that may not be acceptable for the application. In the case of discrete variables, the probability distribution functions can be represented in a tabular form. It should be noted that in the case where the model consists of only continuous variables with linear Gaussian relationships, these algorithms may also be used for inference, but since it can be shown that in this case these algorithms are equivalent to the Kalman Filter, the Kalman Filter is used as the example algorithm.

When the physiology model consists of both continuous and discrete ISVs with nonlinear relationships between the variables, no exact inference solution is possible. In this case, the physiology observer module 122 may use an approximate inference scheme that relies on sampling techniques. The simplest version of this type of algorithm is a Particle Filter algorithm, which uses Sequential Importance Sampling. Markov Chain Monte Carlo (MCMC) Sampling methods may also be used for more efficient sampling. Given complex and non-linear physiologic relationships, this type of approximate inference scheme affords the most flexibility. A person reasonably skilled in the relevant arts will recognize that the model and the inference schemes employed by the physiology observer module may be any combination of the above described or include other equivalent modeling and inference techniques.

When using particle filtering methods, a resampling scheme is necessary to avoid particle degeneracy. The physiology observer may utilize an adaptive resampling scheme. As described in detail below, regions of the ISV state space may be associated with different patient states, and different levels of hazard to the patient. The higher the number, the more hazardous that particular condition is to the patient's health. In order to ensure accurate estimation of the probability of a particular patient condition, it may be necessary to have sufficient number of sampled particles in the region. It may be most important to maintain accurate estimates of the probability of regions with high hazard level and so the adaptive resampling approach guarantees sufficient particles will be sampled in high hazard regions of the state space. FIG. 7 illustrates an example of this resampling based on two internal state variables (“ISV-X” and “ISV-Y”), which may be any internal state variables of the patient, including without limitation any of the internal state variables described herein. State 1 and State 2 have the highest hazard level. The left plot depicts the samples generated from the standard resampling. Notice there are naturally more particles in state 1 and state 2 region because these states are most probable. The right plot shows the impact of the adaptive resampling. Notice how the number of samples in the areas of highest risk has increased significantly. FIG. 35A, described further below, illustrate an example of creating the Conditional Likelihood Kernel 230 in the context of “HLHS Stage 1” example, (where “HLHS” is Hypoplastic Left Heart Syndrome”).

As described in connection with FIG. 35A, in the observation model 221 inference over the DBN is performed using an Extended Kalman Filter (“EKF”), which is a variant of the Kalman Filter that extends the inference engine algorithm for use on applications where the underlying models have nonlinear relationships. The extension is accomplished using a Taylor-series expansion of the nonlinear relationships of the model. This approximation allows the algorithm analytically calculate a Gaussian approximation to the posterior density given the measurements provided to the system. FIG. 35B depicts this Gaussian approximation for P(ISVs(tₖ₊₁)|M(tₖ₊₁)). The depicted density is a multivariate Gaussian that can be fully represented using the conditional mean of the ISVs at the current time, [symbol] (tₖ₊₁|tₖ₊₁), and the conditional covariance matrix of the ISVs, Σ(tₖ₊₁|tₖ₊₁) These quantities are conditioned on all of the available measurements up to the current time step.

FIG. 35C, depicts the computation steps of the E K F as they relate to the current implementation. In the first step, the density is initialized using assumed initial mean and covariance conditions for the ISVs which can be informed by patient population norms or medical literature. After initialization, the density is passed to the predict step in which the ISV density is predicted forward to the time of the current measurement. This prediction is accomplished by predicting the conditional mean forward in time using the dynamic model specified in the physiology observer module, and by predicting the conditional covariance matrix utilizing a linearization of this dynamic model with the addition of“process noise” to account for uncertainty in the model of the dynamics. Note, this is the same calculation that is described in the predict module of physiology observer module except specifically for Gaussian densities. Because the E K F algorithm has a predict step incorporated in the calculation, so the physiology observer is able to utilize this predict method as part of the processing.

It should be noted that the Extended Kalman Filter, as described above, is not limited to use in creating the Conditional Likelihood Kernel 230 in the context of “HLHS Stage 1.” Rather, the Extended Kalman Filter may be used to create any Conditional Likelihood Kernel 230 supported by this disclosure.

Following the prediction of the PDF to the current measurement time, the posterior density is calculated using Bayes Rule combining the new information provided by the current measurement m(tₖ) with the prior predicted PDF in the update step. Because of the Gaussian approximation, this calculation is analytically tractable and only involves calculating the posterior conditional mean and posterior conditional covariance matrix. Once these quantities have been updated, the entire density can be calculated.

The update step takes as an input the observation model specified in the physiology observer. Using this model, the calculation first calculates the Kalman Gain (K) which determines how much to the posterior conditional mean and covariance matrix change from prior given on the new data. The Kalman Gain is a function of the prior conditional covariance matrix, the expected noise associated with the measurement and a linearization of the observation model provided by the observer. Once the Kalman Gain is computed, the posterior conditional mean is updated from the prior mean using the difference between the measurement value and the expected measured value scaled by this gain. The posterior conditional covariance is updated from the prior in a similar manner, reducing the overall uncertainty proportional to the amount of information that the measurement provides about the underlying ISVs. Following this step, the conditional density is passed back to the predict method where it is predicted to the next (i.e., subsequent) measurement step. It is also returned to the physiology observer module.

### 3. Inference Engine {222}

The inference engine 222 of module 122 achieves this update by using the predicted probability density functions of the ISVs 211 as a-priori probabilities, which are updated with the statistics (i.e., the conditional likelihood kernel 230) of the received measurements from data reception module 121 to achieve the posterior probabilities reflecting the current (at time tₖ₊₁) probability density functions of the ISVs 213. The inference engine 222 accomplishes the update step 220 with the following equation which is Bayes' Theorem,

$$P ( ISVs ( {t}_{k + 1} ) \vert M ( {t}_{k + 1} ) ) = \frac{\begin{matrix}P ( {m}_{1} ( {t}_{k + 1} ) , {m}_{2} ( {t}_{k + 1} ) , … {m}_{n} ( {t}_{k + 1} ) \vert \\ ISVs ( {t}_{k + 1} ) ) P ( ISVs ( {t}_{k + 1} ) \vert M ( {t}_{k} ) )\end{matrix}}{P ( {m}_{1} ( {t}_{k + 1} ) , {m}_{2} ( {t}_{k + 1} ) , … {m}_{n} ( {t}_{k + 1} ) \vert M ( {t}_{k} ) )}$$

where:
P(ISVs(tₖ₊₁)|M(tₖ₊₁)) are the “posterior probabilities” at time step tₖ₊₁ expressed as conditional probabilities;
P(m₁(tₖ₊₁), m₂(tₖ₊₁), . . . mₙ(tₖ₊₁)|ISVs(tₖ₊₁)) is the conditional likelihood kernel provided by the observation model 221 that determines how likely the currently received measurements are given the currently predicted ISVs;
P(ISVs(tₖ₊₁)|M(tₖ)) are the predicted PDFs of ISVs 211 produced in the predict model at the previous time step tₖ (see, e.g., FIG. 2B); and

P(m₁(tₖ₊₁), m₂(tₖ₊₁), . . . mₙ(tₖ₊₁)|M(tₖ)) is the predicted PDFs of the measurements received at the time step given the measurements received up to that time step.

At the initialization time (e.g., t=0 or t=tᵢₙᵢₜ) when no then-current estimate of probability density functions of the ISVs is available, the physiology observer module 122 may utilize initial estimates 240, which may be derived from an educated guess of possible values for the ISVs or statistical analysis of previously collected patient data.

Referring now to FIG. 2A, it can be seen that the output of the Inference Engine 222 at time step tₖ₊₁ (i.e, the “posterior probabilities” of time step tₖ₊₁) is sent in two directions.

In the first direction, the output of the Inference Engine 222 is provided to the predict module 210, where it may be referred-to as the “current estimates of PDFs of ISVs” 213 (see, e.g., FIG. 2B and its related description).

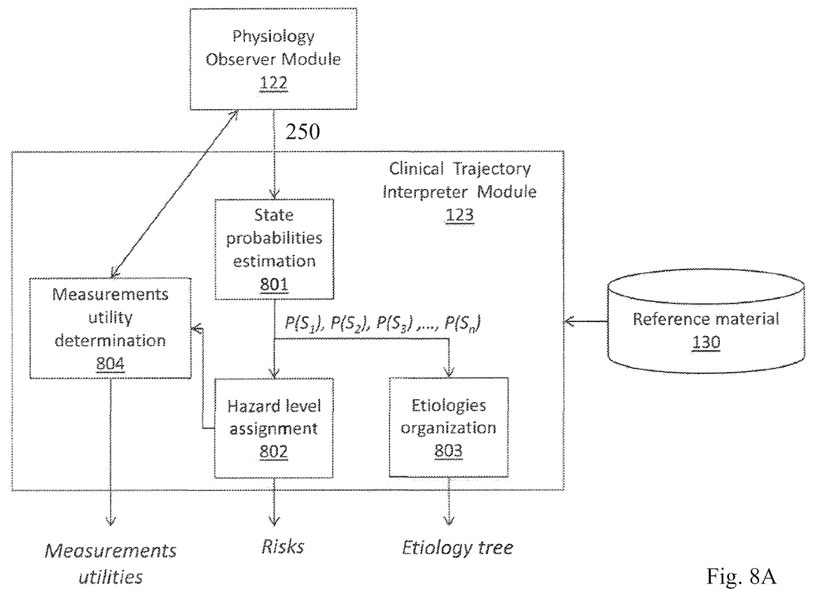

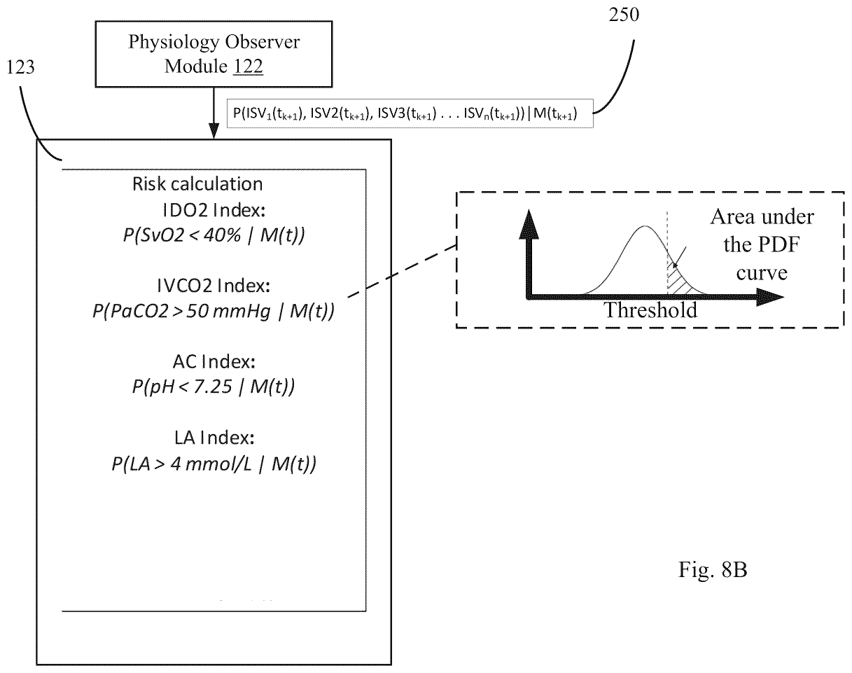

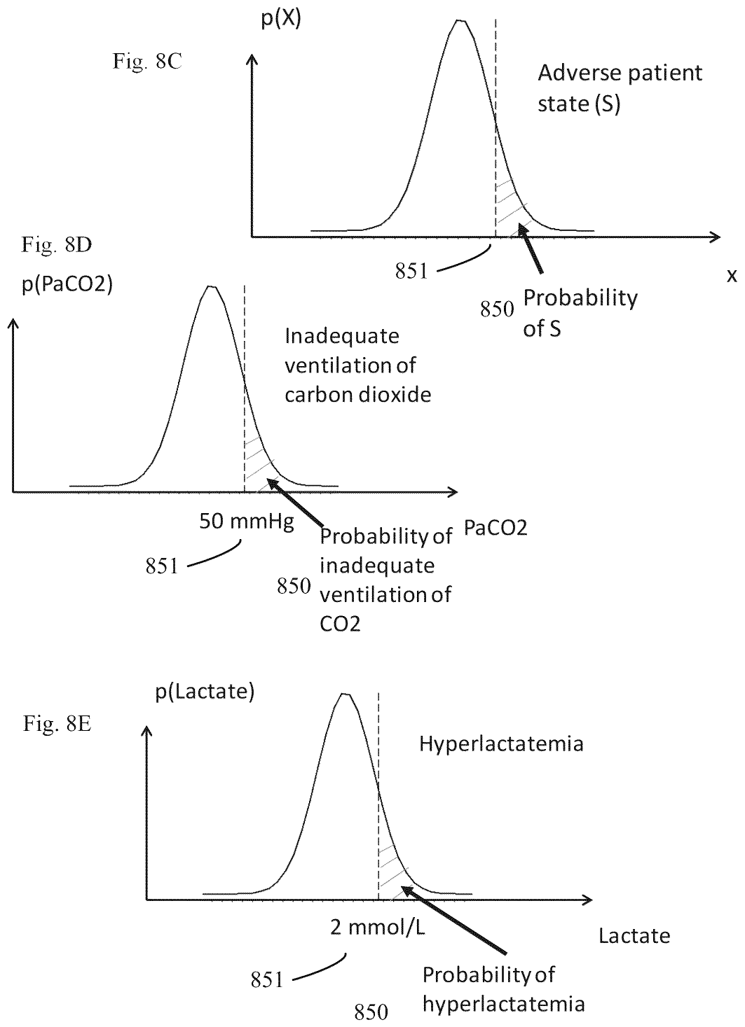

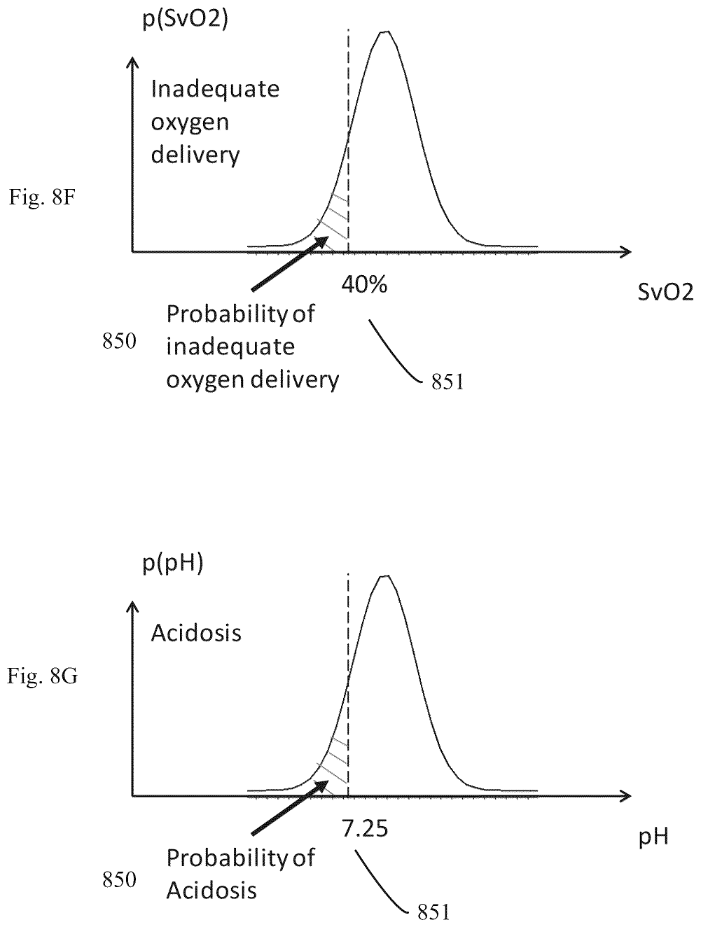

In the second direction, the output of the Inference Engine 222 at time step tₖ₊₁ (expressed in the figures simply as probabilities) is provided to the to the clinical trajectory interpreter module 123, where that output may be referred-to as the “joint Probability Density Functions of the ISVs from the physiology observer module”) 250. Operation of the to the clinical trajectory interpreter module 123 is described further herein (see, e.g., FIG. 2C and its related description; FIG. 8A and FIG. 8B).

### II. Clinical Trajectory Interpreter Module {123} {Determining Patient States}

Using the posterior probabilities (250) from the Inference Engine 222 for time step tₖ₊₁, the Clinical Trajectory Interpreter Module 123 performs state probability estimation 801 to calculate the probabilities of different patient states.

Referring now to FIG. 8A, the Clinical Trajectory Interpreter 123 takes the joint Probability Density Functions of the ISVs 250 from physiology observer module 122, and performs state probability estimation 801 to calculate the probabilities of different patient states. The Probability Density Functions of the ISVs may be defined in closed form, for example multidimensional Gaussians 260, or approximated by histogram 280 of particles 270, as illustrated in FIG. 2D, FIG. 2E and FIG. 2F. In both cases, the probability density functions of the ISVs can be referred to as: (ISV1(t), ISV2(t), . . . , ISVn(t)), where tis the time they refer to.

FIG. 8A may refer to the output 250 of the Inference Engine 222 as “the joint Probability Density Functions of the ISVs from the physiology observer module.” This data may be represented in a variety of ways: the Probability Density Functions of the ISVs may be defined in closed form, for example multidimensional Gaussians 260, or approximated by histogram 280 of particles 270, as illustrated in FIG. 2D, FIG. 2F and FIG. 2G.

Determining the patient states (i.e, “determining the probability of the patient being in a particular state Sᵢ”) may be done in a variety of ways.

Generally, the PDFs of the ISVs define a domain. In illustrative embodiments, the domain is partitioned into quadrants, each quadrant representing a patient state. The probability that the patient is in a given one of the four patient states is determined by the quantity of the PDFs of the ISV's located within the given quadrant.

This may be described as:
(i) partitioning a domain spanned by the internal state variables into different regions, each region defining a separate patient state; and
(ii) integrating the probability density functions over the regions corresponding to each particular patient state to produce probabilities that the patient may be classified under each of said possible patient states.

### FIG. 2B: Multidimensional Gaussian 260

Where the data is in the form of a multidimensional Gaussian 260, integration may be performed directly:

$$P ( {S}_{i} ( t ) ) = {\int }_{- ∞}^{∞} … {\int }_{- ∞}^{∞} P ( S \vert {ISV}_{1} , {ISV}_{2} , … , {ISV}_{n} ) P ( {ISV}_{1} ( t ) , {ISV}_{2} ( t ) , … , {ISV}_{n} ( t ) ) {dISV}_{1} … {dISV}_{n}$$

### FIG. 2E and FIG. 2F: Histogram of Particles 270

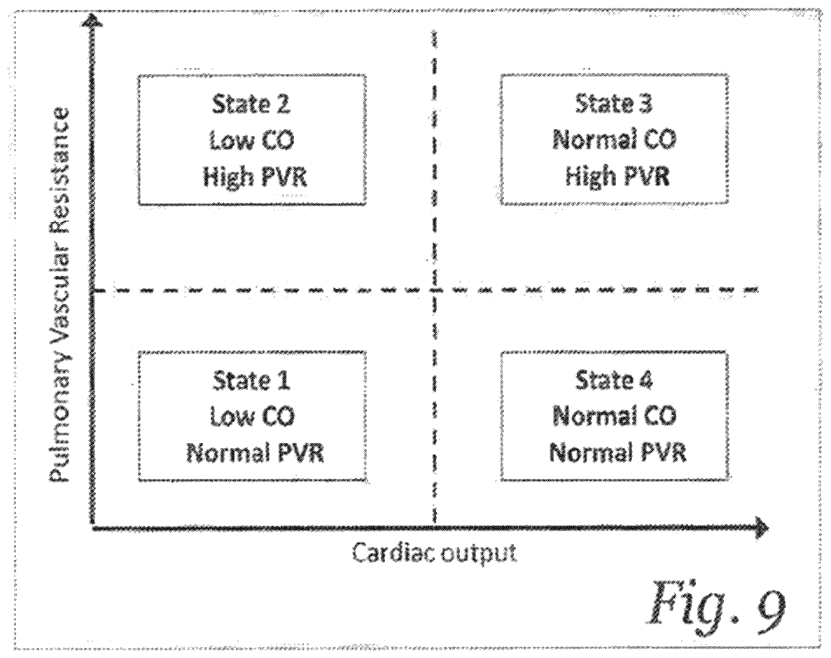

In case that the output 250 of the Inference Engine 222 is approximated by a histogram 280 of particles 270 and P(S|ISV₁, ISV₂, . . . , ISVₙ) is defined by a partition of the space spanned by ISV₁, ISV₂, . . . , ISVₙ into regions as shown in FIG. 9, the probability P(Sᵢ(t)) may be calculated by calculating the fraction of particles 270 in each region.

### Example: FIG. 9

FIG. 9 illustrates a non-limiting example of a definition of a patient state that may be employed by the clinical trajectory interpreter module 123. Specifically, it assumes that the function P(S|ISV₁, ISV₂, . . . , ISVₙ) may be defined by partitioning the domain spanned by the internal state variables ISV₁, ISV₂, . . . , ISVₙ. The particular example assumes that the patient physiology is described by two internal state variables: Pulmonary Vascular Resistance (PVR) and Cardiac Output (CO). The particular risks and respective etiologies that may be captured by these two ISVs emanate from the effects of increased pulmonary vascular resistance on the circulation. Specifically, high PVR may cause right-heart failure and consequently reduced cardiac output. Therefore, PVR can be used to define the attributes of Normal PVR and High PVR, and CO to define the attributes of Normal CO and Low CO, by assigning thresholds with the two variables. By combining these attributed, four separate states can be defined: State 1: Low CO, Normal PVR; State 2: Low CO, High PVR; State 3: Normal CO, High PVR; State 4: Normal CO Normal PVR.

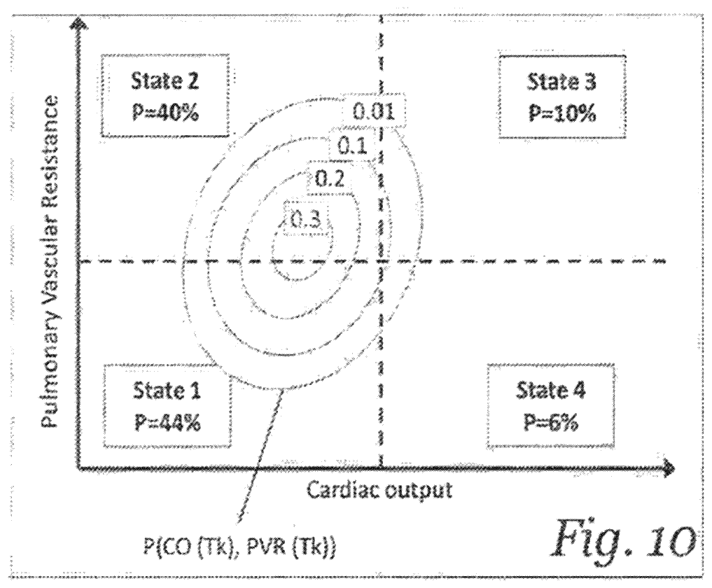

### Example: FIG. 10

FIG. 10 illustrates a non-limiting example of how the clinical trajectory interpreter module 123 may employ the definition of patient states to assign probabilities that the patient may be classified under each of the four possible patient states at a particular point of time. In the example, the clinical trajectory interpreter module 123 takes the joint probability density function of P(Cardiac Output (Tk), Pulmonary Vascular Resistance (Tk)) and integrates it over the regions corresponding to each particular state, which produces P(S1(Tk)), P(S2(Tk)), P(S3(Tk)), and P(S4(Tk)). In this way, the clinical trajectory interpreter module 123 assigns a probability that a particular patient state is ongoing, given the information provided by the physiology observer module 122. Note that if the output of the physiology observer module 122 is not a closed form function 260 but a histogram 280 of particles 270, the clinical interpreter will not perform integration but just calculate the relative fraction of particles 270 within each region.

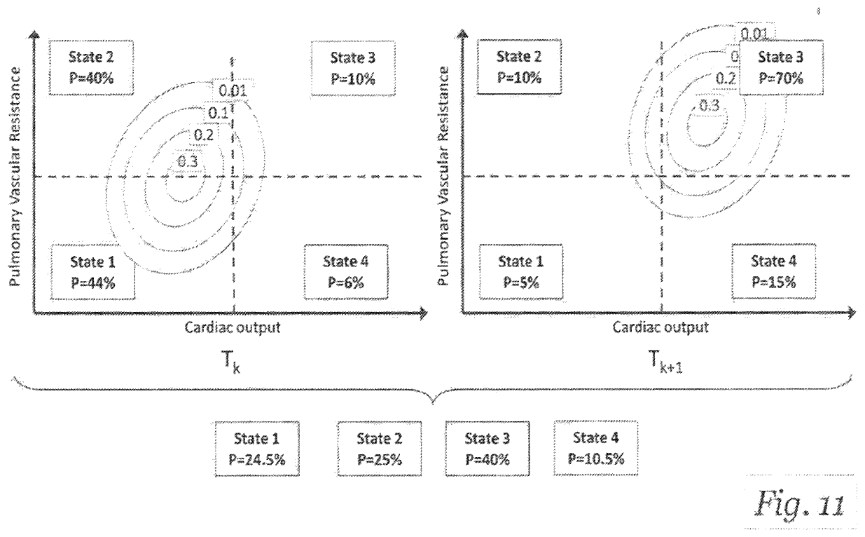

### Example: FIG. 11

FIG. 11 illustrates an alternative approach of estimating the probabilities for different patient states. In this alternative approach, to calculate the probabilities P(S1), P(S2), P(S3) and P(S4), the clinical trajectory interpreter module 123 employs the joint probability functions of the ISVs for two consecutive time windows Tₖ and Tₖ₊₁ to calculate a moving window average. Note in the example that the size of the window is doubled for two time instances, which indicates that the window may be of an arbitrary, suitable size. As a result of this moving window averaging, the clinical trajectory interpreter module 123 performs a dynamic analysis of the trajectory of the ISVs. That is, it gives a metric of the probability that the physiology trajectory, as described by the ISVs, may be found in a particular region in a particular time frame. In other words, this probability calculation gives an estimate of the probability that a particular patient state may be ongoing in the chosen time-frame, as opposed to just at a chosen time instance.

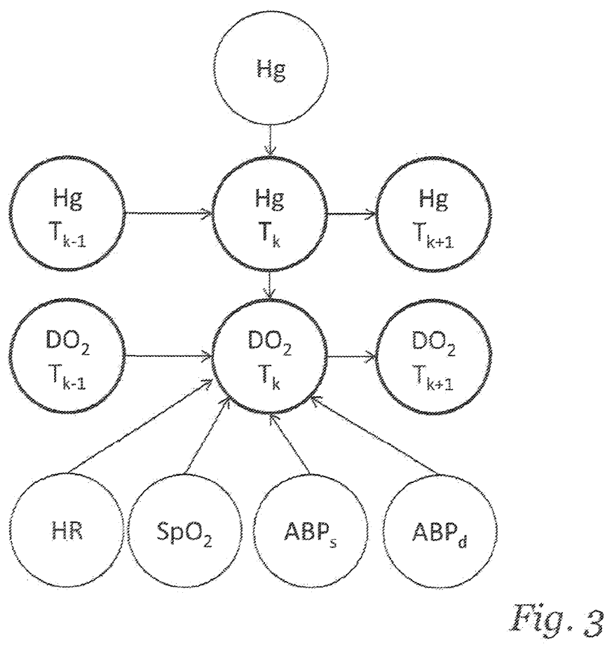

FIG. 3 illustrates a non-limiting example of models that enable the physiology observer in accordance with the present disclosure. While not directly observable, the management of oxygen delivery, DO2, is an important part of critical care. Therefore, precise estimation of DO2 can inform improved clinical practice. In the illustrated example, this estimation is achieved through the measurements of hemoglobin concentration (Hg), heart rate (HR), diastolic and systolic arterial blood pressures, and SpO2. The dynamic model 212 assumes that oxygen delivery is driven by a feedback process which stabilizes it against stochastic disturbances. Similarly, hemoglobin concentration is controlled around the norm value of 15 mg/dL. The observation model 221 takes into account the relationship between arterial oxygen saturation SpO2, hemoglobin concentration and arterial oxygen content CaO2, the dependence of the difference between systolic, ABPs, and diastolic, ABPd, arterial blood pressures (also called pulse pressure) on the stroke volume, and the relationship between heart rate, HR, stroke volume, SV, and cardiac output. The two models are abstracted as a Dynamic Bayesian Network (DBN), and the physiology observer module 122 utilizes the DBN to continuously track the oxygen delivery. A Dynamic Bayesian Network is a systematic way to represent statistical dependencies in terms of a graph whose vertices signify variables (observable and unobservable), and whose edges show causal relationships. Further descriptions of an exemplary DBN for DO2 estimation can be found in U.S. Provisional Application No. 61/699,492, filed on Sep. 11, 2012, entitled SYSTEMS AND METHODS FOR EVALUATING CLINICAL TRAJECTORIES AND TREATMENT STRATEGIES FOR OUTPATIENT CARE, Attorney Docket No. 3816/10301, and U.S. Provisional Application No. 61/684,241, filed on Aug. 17, 2012, entitled SYSTEM A N D METHODS FOR PROVIDING RISK ASSESSMENT IN ASSISTING CLINICIANS WITH EFFICIENT AND EFFECTIVE BLOOD MANAGEMENT, Attorney Docket No. 3816/10101, to which priority is claimed, the disclosure of which is incorporated herein by reference.

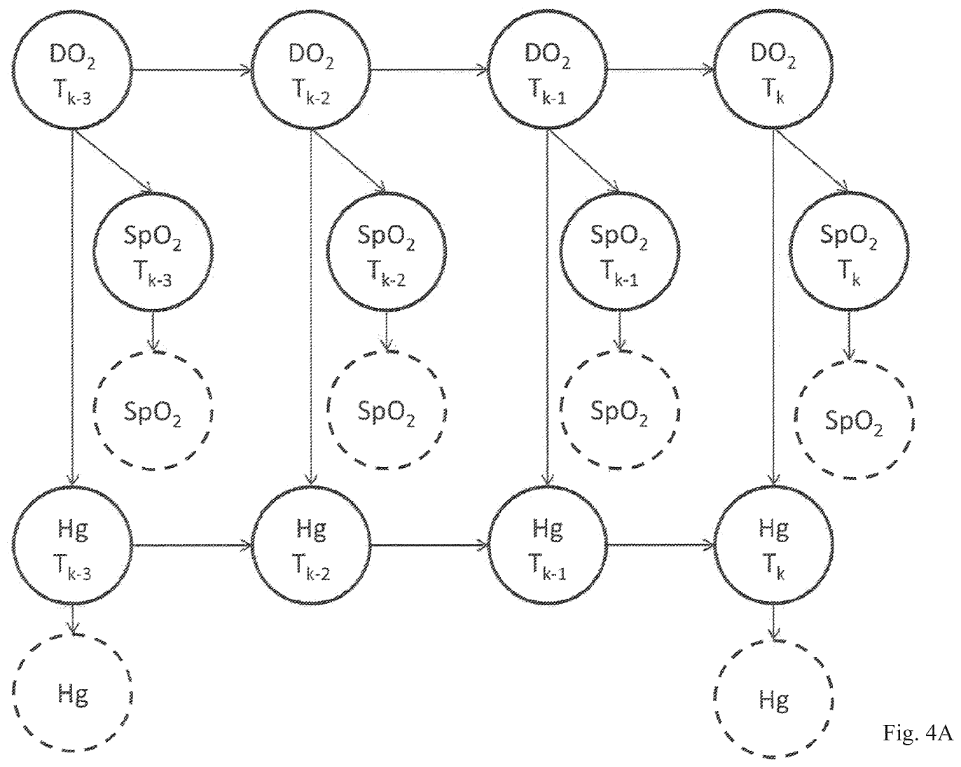

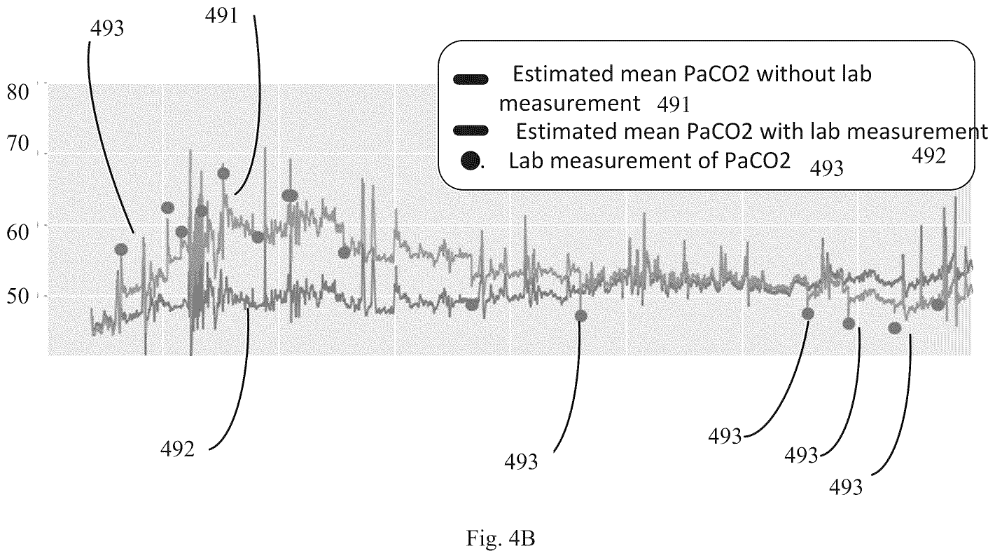

FIG. 4A depicts a non-limiting example of the physiology observer described above tracking DO2, but over a longer time interval, i.e., four (4) time steps. In the observer, the main hidden ISV is the oxygen delivery variable (DO2). The two types of measurements, Hemoglobin (Hg) and oximetry (SpO2) are in dashed circles in FIG. 4A. SpO2 is an example of the continuous or periodic measurements that the physiology observer module 122 receives from sensors, such as bedside monitors 102 and treatment devices 104 connected to the patient 101 that continuously report information. Hemoglobin (Hg) is an example of an intermittent or aperiodic measurement extracted from patient lab work that is available to the observer on a sporadic and irregular basis, and latent at times, relative to current system time. The physiology observer module 122 is capable of handling both types of measurements because, along with tracking the hidden ISVs, e.g. DO2, module 122 also continuously maintains estimates of the observed values for all types of measurements, even when measurements are not present. FIG. 4A depicts these estimates for the case of SpO2 and Hg. A s can be seen, the SpO2 measurements are available regularly at each time step, whereas Hg is only available at two of the time steps.

FIG. 4B illustrates a method applying intermittent laboratory data through the physiology observer module to achieve better accuracy in an estimated ISV PDF. The specific example shows what the estimated mean of the PDF of the PaCO2 ISV is without incorporating arterial blood gases that directly measure this internal state variable, and how this estimate changes as arterial blood gas measurements are introduced into the system. Specifically, the estimated mean of the PaCO2 ISV is much closer to the actual measured PaCO2 when these measurements are incorporated as inputs.

This is achieved as follows:
The physiology observer module includes a hidden ISV called alveolar dead space which takes into account that lung ventilation may not be efficiently removing CO2 from the blood. I.e. the higher the alveolar dead space is, the higher the difference is between expired CO2 as measured by end-tidal CO2 measurement (EtCO2) and arterial CO2 as measured by PaCO2 arterial blood gases.
The PDF of this ISV is used to predict various measurements acquired from the patient, some of which might include minute ventilation, end-tidal CO2, and PaCO2 arterial blood gases
As a non-limiting example, if minute ventilation and end-tidal CO2 are continuously acquired measurements at every time tₖ₊₁, the inference engine can use Bayes theorem to update the PDF of the various ISVs, one of which is PaCO2. That is, in the formula,

$$P ( ISVs ( {t}_{k + 1} ) \vert M ( {t}_{k + 1} ) ) = \frac{P ( {m}_{1} ( {t}_{k + 1} ) , {m}_{2} ( {t}_{k + 1} ) \vert ISVs ( {t}_{k + 1} ) ) P ( ISVs ( {t}_{k + 1} ) \vert M ( {t}_{k} ) )}{P ( {m}_{1} ( {t}_{k + 1} ) , {m}_{2} ( {t}_{k + 1} ) \vert M ( {t}_{k} ) )}$$

m₁ and, m₂ are measured values of EtCO2 and minute ventilation.
When a PaCO2 blood gas measurement is acquired at next time tₖ+2 in conjunction with measurements of end-tidal EtCO2 and minute ventilation, the measurement vector is augmented with the PaCO2 measurement and the above formula becomes:

$$P ( ISVs ( {t}_{k + 2} ) \vert M ( {t}_{k + 2} ) ) = \frac{P ( {m}_{1} ( {t}_{k + 2} ) , {m}_{2} ( {t}_{k + 2} ) , {m}_{3} ( {t}_{k + 2} ) \vert ISVs ( {t}_{k + 2} ) ) P ( ISVs ( {t}_{k + 2} ) \vert M ( {t}_{k + 1} ) )}{P ( {m}_{1} ( {t}_{k + 2} ) , {m}_{2} ( {t}_{k + 2} ) , {m}_{3} ( {t}_{k + 2} ) \vert M ( {t}_{k + 1} ) )}$$

Where again m₁ and, m₂ are measured values of EtCO2 and minute ventilation, and m₃ is the PaCO2 measurement.
This additional information is utilized in two ways. First, the uncertainty in the PaCO2 ISV PDF is reduced (See FIG. 4B) given the more accurate direct observation of PaCO2 provided by the arterial blood gas. Second, since the PaCO2 and EtCO2 measurements are observed simultaneously, the combined information is used by the physiology observer to estimate alveolar dead-space more precisely.
When the new estimated PDF of alveolar dead-space is propagated forward in time by the dynamic model, the accuracy of the estimated PaCO2 PDF is improved even when m₃, the PaCO2 measurement, is not present.

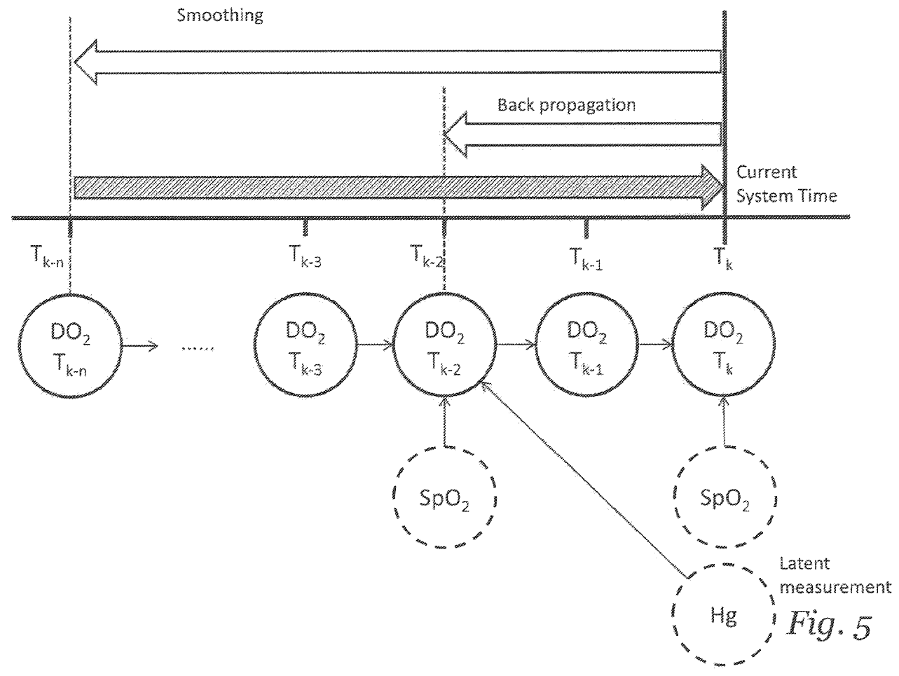

As mentioned above, certain measurements, such as Hemoglobin, are available to the system with an unknown amount of time latency, meaning the measurements are valid in the past relative to the current time and the time they arrive over the data communication links. The physiology observer module 122 may handle such out of sequence measurements using back propagation, in which the current estimates of the ISVs are projected back in time to the time of validity of the measurements, so that the information from the latent measurement can be incorporated correctly. FIG. 5 depicts such time line. In FIG. 5, hemoglobin arrives at the current system time, tₖ, but is valid and associated back to the ISV (DO2) at time Tₖ₋₂. Back propagation is the method of updating the current ISVs probability estimates P(ISVs(tₖ)|M(tₖ)) with a measurement that is latent relative to the current time, m(tₖ₋ₙ). Back propagation is accomplished in a similar manner to the prediction method described previously. There is a transition probability kernel, P(ISVs(tₖ₋ₙ)|ISVs(tₖ)), that defines how the current probabilities evolve backwards in time. This can then be used to compute probabilities of the ISVs at time tₖ₋ₙ given the current set of measurements which excludes the latent measurement, as follows:

$$P ( ISVs ( {t}_{k - n} ) \vert M ( {t}_{k} ) ) = {\int }_{ISVs ∈ ISV} P ( ISVs ( {t}_{k - n} ) \vert ISVs ( {t}_{k} ) ) P ( ISVs ( {t}_{k} ) \vert M ( {t}_{k} ) ) dISVs$$

Once these probabilities are computed, the latent measurement information is incorporated using Bayes' rule in the standard update:

$$P ( ISVs ( {t}_{k - n} ) \vert M ( {t}_{k} ) , m ( {t}_{k - n} ) ) = \frac{P ( m ( {t}_{k - n} ) \vert ISVs ( {t}_{k - n} ) ) P ( ISVs ( {t}_{k - n} ) \vert M ( {t}_{k} )}{P ( M ( {t}_{k} ) , m ( {t}_{k - n} ) )}$$

The updated probabilities are then propagated back to the current time tₖ using the prediction step described earlier. Back propagation can be used to incorporate the information.

Another functionality of the physiology observer module 122 includes smoothing. The care provider using the system 100 may be interested in the patient state at some past time. With smoothing, the physiology observer module 122 may provide a more accurate estimate of the patient ISVs at that time in the past by incorporating all of the new measurements that the system has received since that time, consequently providing a better estimate than the original filtered estimate of the overall patient state at that time to the user, computing P(ISVs(t_{k_n})|M(tₖ)). This is accomplished using the first step of back propagation in which the probability estimates at time tₖ which incorporate all measurements up to that time are evolved backwards to the time of interest tₖ₋ₙ using the defined transition probability kernel. This is also depicted in FIG. 5, in which the user is interested in the patient state at tₖ₋ₙ and the estimates are smoothed back to that time.

Because physiology observer module 122 maintains estimates of each of the measurements available to the system 100 based on physiologic and statistical models, module 122 may filter artifacts of the measurements that are unrelated to the actual information contained in the measurements. This is performed by comparing the newly acquired measurements with the predicted likelihoods of probable measurements given the previous measurements. If the new measurements are considered highly unlikely by the model, they are not incorporated in the estimation. The process of comparing the measurements with their predicted likelihoods effectively filters artifacts and reduces noise. FIG. 6 shows an example of such a process involving an internal state variable (“ISV1”), which may be any internal state variable of the patient, including any internal state variable described herein. For example, the internal state variable in some embodiments may be mean arterial blood pressure (ABPm). Because ABPm is collected using an intravenous catheter, the measured signals are often corrupted with artifacts that result in incorrect measurements when the catheter is used for medical procedures such as blood draws or line flushes. FIG. 6 shows the raw ISV (ABPm) measurements prior to being processed by the physiology observer with the measurement artifacts identified, as well as the filtered measurements after being processed by the physiology observer module 122. As can be seen, the measurement artifacts have been removed and the true signal is left.

### Clinical Trajectory Interpreter

Referring now to FIG. 8A and FIG. 8B, the Clinical Trajectory Interpreter 123 takes the joint Probability Density Functions of the ISVs from physiology observer module 122, and performs state probability estimation 801 to calculate the probabilities of different patient states. The Probability Density Functions of the ISVs may be defined in closed form, for example multidimensional Gaussians 260, or approximated by histogram 280 of particles 270, as illustrated in FIGS. 2B-D. In both cases, the probability density functions of the ISVs can be referred to as: P(ISV1(t), ISV2(t), . . . , ISVn(t)), where t is the time they refer to. Given the internal state variables the patient state may be defined by a conditional probability density function:
P(S|ISV1, SV2, . . . , ISVn), where S∈S₁, S₂, . . . , S_{N} represents all possible patient states Sᵢ

Then determining the probability of the patient being in a particular state S may be performed by the equation:

$$P ( {S}_{i} ( t ) ) = {\int }_{- ∞}^{∞} … {\int }_{- ∞}^{∞} P ( S \vert {ISV}_{1} , {ISV}_{2} , … , {ISV}_{n} ) P ( {ISV}_{1} ( t ) , {ISV}_{2} ( t ) , … , {ISV}_{n} ( t ) ) {dISV}_{1} … {dISV}_{n}$$

In case that P(ISV1(t), ISV2(t), . . . , ISVn(t)) is defined by a closed form function such as multidimensional Gaussian 260, the integration may be performed directly. In case that P(ISV1(t), ISV2(t), . . . , ISVn(t) is approximated by a histogram 280 of particles 270 and P(S|ISV₁, ISV₂, . . . , ISVₙ) is defined by a partition of the space spanned by ISV₁, ISV₂, . . . , ISVₙ into regions as shown in FIG. 9, the probability P(Sᵢ(t)) may be calculated by calculating the fraction of particles 270 in each region.

Once patient state probabilities are estimated, the clinical trajectory interpreter module 123 may assign different hazard levels 802 for each patient state or organize the states into different etiologies 803. The clinical trajectory interpreter module 123, in conjunction with the physiology observer module 122, may perform measurements utility determination 804 to determine the utility of different invasive measurements such as invasive blood pressures or invasive oxygen saturation monitoring. In one embodiment, the Clinical trajectory interpreter Module 123 determines the probabilities that the patient is in a particular state, rather than the exact state that the patient is in.

FIG. 8B illustrates an embodiment of a Clinical Trajectory Interpreter 123 module that may be referred-to as a Risk Calculation module. The Risk Calculation depicted calculates the probability that particular internal state variables that represent key bio-markers, e.g. SvO2 or PaCO2, are abnormal, i.e above or below particular pre-defined clinically significant values at a particular time (e.g., in keeping with illustrative embodiments, time tₖ₊₁). Specifically, the Risk calculation takes as an input, the continuous probability densities estimated by the Physiology observer module for a particular time step, and calculates the cumulative probabilities of interest. The four probabilities currently calculated are 1) the probability of inadequate oxygen delivery defined as a mixed venous oxygen saturation (SvO2) below 40%, also referred to as the IDO2 Index, 2) the probability of inadequate ventilation of carbon dioxide, defined as arterial partial pressure greater than 50 mmHg, also referred to as the IV CO2 Index, 3) the probability of acidosis, defined as blood pH below 7.25, also referred to as the AC Index, and 4) the probability of hyperlactatemia, defined as lactate blood levels greater than 4.0 mmol/L, also referred to as the LA Index.

Various patients states (adverse medical conditions), their associate internal state variables, and sensors included in a set of sensors supplying patient measurements to the system 100 are listed below.

| Patient State | Hidden ISV | Sensor(s) |
|---|---|---|
| Inadequate Oxygen Delivery | mixed venous | a heart rate sensor and |
| | oxygen saturation | an SpO2 sensor, |
| Inadequate ventilation of | arterial partial | a heart rate sensor and |
| carbon dioxide (IV CO2 Index) | pressure of carbon | an SpO2 sensor, |
| defined as a patient's arterial | dioxide blood | respiratory rate sensor |
| partial pressure of carbon | PaCO2 | |
| dioxide blood being greater | | |
| than a particular value, e.g. 50 | | |
| mmHg | | |
| Acidosis (A C Index) defined as | Arterial blood pH | a heart rate sensor and |
| a patient's blood pH being less | | an SpO2 sensor, |
| than a particular value, e.g. | | respiratory rate sensor |
| 7.25 | | |
| Hyperlactatemia (LA Index) | arterial lactate level | a heart rate sensor and |
| defined as a patient's arterial | | an SpO2 sensor |
| lactate level being greater than | | |
| a particular value, e.g. 4 | | |
| mmol/L | | |

As a non-limiting example, the patient state of inadequate oxygen delivery may be inferred by the invention from a heart rate and an SpO2 sensor in the following ways. The physiology observer module 122 continuously interprets ISV data based on the following understandings:
If a patient has decreasing pulse oximetry and rising heart rate the physiology model we use will infer that there is a determinable probability of rising lactate.
The model by the physiology observer in this example will capture explicitly the relationship between the ISVs of arterial saturation (measured by SpO2) and heart rate (measured by the heart rate sensor) and the hidden ISV of mixed venous oxygen saturation.
As a result, at each time instance the physiology observer will update the PDF ISV of mixed venous oxygen saturation, and because of the inferred increase in heart rate and decrees in arterial saturation, the PDF will imply higher probability for lower values of the ISV SvO2.
This in turn will be interpreted by the Clinical Interpreter module as a rising risk for inadequate oxygen delivery reflected by the calculation:

$$IDO 2 Index = P ( SvO 2 < 40 \% \vert M ( {t}_{k} ) ) = {\int }_{- ∞}^{40} P ( SvO 2 \vert M ( {t}_{k} ) ) dSvO 2$$

Note that in the foregoing formula, the threshold is 40 percent, but the that illustrative embodiment does not limit all embodiments. The threshold may be determined by the clinician or system developer or operator.

Similarly, another non-limiting example is how the state of Hyperlactatemia can be calculated with the same set of sensors: i.e.:
The model in the physiology observer can relate the state of inadequate oxygen delivery as reflected by the ISV of SvO2 as the probable onset of anaerobic metabolism.
The model that will take into account that the more probable is that the patient is experiencing anaerobic metabolism the higher is the likelihood of lactate production, which will mean that with each update the PDF of the ISV of lactate will indicate higher probable values for lactate.

In turn the clinical trajectory interpreter module will compute the risk for the state of hyperlactatemia as:

$$LA Index = P ( Lactate < 4 \frac{m m o l}{L} \vert M ( {t}_{k} ) ) = {\int }_{- ∞}^{4} P ( Lactate \vert M ( {t}_{k} ) ) dLactate$$

Note that in the foregoing formula, the threshold is 4 mmol/L, but the that illustrative embodiment does not limit all embodiments. The threshold may be determined by the clinician or system developer or operator.

Similarly, yet another non-limiting example is using respiratory rate sensor in addition to SpO2 and Heart rate sensors to determine the probability that the patient is in a state of inadequate ventilation of carbon dioxide. In this example a rising respiratory rate and heart rate while arterial saturation stays the same may be interpreted by the model in the physiology observer as a physiologic response the probable elevation of arterial carbon dioxide. This inference will be reflected by higher probable values of the ISV of PaCO2, and the risk for the patient being in inadequate ventilation of carbon dioxide state can then be computed by:

$$IVCO 2 Index = P ( PaCO 2 > 50 mmHg \vert M ( {t}_{k} ) ) = {\int }_{50}^{∞} P ( PaCO 2 \vert M ( {t}_{k} ) ) dPaCO 2$$

Note that in the foregoing formula, the threshold is 50 mmHg, but the that illustrative embodiment does not limit all embodiments. The threshold may be determined by the clinician or system developer or operator.

Finally another example is the computation of probability that a patient is in the state of acidosis. Acidosis can be caused both by rising PaCO2 or rising lactate. A n additional effect in the model of the physiology observe which captures this relationship can then infer the rising probable values of the ISVs of lactate and PaCO2 as decreasing probable values of arterial pH as captured by the PDF of this ISV. As a result the probability of the state of acidosis can be given by:

$$AC Index = P ( pH < 7.25 \vert M ( {t}_{k} ) ) = {\int }_{- ∞}^{7.25} P ( pH \vert M ( {t}_{k} ) ) dpH$$

Note that in the foregoing formula, the threshold is 7.25, but the that illustrative embodiment does not limit all embodiments. The threshold may be determined by the clinician or system developer or operator.

The variables SvO2, PaCO2, pH, and Lactate are all internal state variables, or related to internal state variables, for which the Physiology observer calculates the probability density of. Note, in FIG. 8B, the dashed call-out box graphically depicts an illustrative example of this calculation. FIG. 8C schematically illustrates a generic embodiment of this calculation, for a PDF of an ISV termed “p(X).” The probability 850 of adverse patient state (S) (e.g., an adverse medical condition) is defined as the area under the curve of p(X) above (or in some embodiments, below) a threshold 851. M ore specific embodiments are presented in FIG. 8D, FIG. 8E, FIG. 8F and FIG. 8G, discussed below. In general, the threshold in each such embodiments may be specified by the clinician (e.g., doctor, nurse, etc.) using the system 100, based for example on which adverse medical condition is suspected by the clinician.

It should be noted that each of the probabilities can be calculated from densities either conditioned on contemporaneous measurement data (e.g., measurements received for time step tₖ₊₁) (as shown in the equations) or not conditioned on measurement data, allowing the system to produce these quantities regardless data availability levels. The calculations can be performed via standard numerical integration techniques, or when the functional form of the underlying densities is more complicated, Monte Carlo integration techniques can be used. In the current implementation, the densities are Gaussian and so standard software packages for computing these quantities are available.

Once calculated, these Risk quantities are sent to the Display and notification system module 124 for display on a display device.

### Examples

FIG. 8D illustrate the evaluation of the state of Inadequate Ventilation of Carbon Dioxide based on the ISV of partial pressure of arterial blood CO2 (PaCO2). A s a non-limiting example the probability 850 of this state is computed as the cumulative distribution of p(PaCO2) greater than the 50 mmHg threshold. The resulting Clinical Risk can be displayed as an index whose instantaneous time value is given by:

$$IVCO 2 Index = P ( PaCO 2 > 50 mmHg \vert M ( {t}_{k} ) ) = {\int }_{50}^{∞} P ( PaCO 2 \vert M ( {t}_{k} ) ) dPaCO 2$$

FIG. 8E illustrate the evaluation of the state of Hyperlactatemia based on the ISV of whole blood Lactate. As a non-limiting example the probability 850 of this state is computed as the cumulative distribution of whole blood Lactate [p(Lactate)] being above a 2 mmol/L threshold, or in some embodiments, a 4 mmol/L threshold. The resulting Clinical Risk can be displayed as an index whose instantaneous time value is given by:

$$LA Index = P ( Lactate < 4 \frac{m m o l}{L} \vert M ( {t}_{k} ) ) = {\int }_{- ∞}^{4} P ( Lactate \vert M ( {t}_{k} ) ) dLactate$$

FIG. 8F illustrate the evaluation of the state of Inadequate Oxygen Delivery based on the ISV of mixed venous oxygen saturation (SvO2). As a non-limiting example the probability 850 of this state is computed as the cumulative distribution of p(SvO2) less than a 40% threshold. The resulting Clinical Risk can be displayed as an index whose instantaneous time value is given by:

$$IDO 2 Index = P ( SvO 2 < 40 \% \vert M ( {t}_{k} ) ) = {\int }_{- ∞}^{40} P ( SvO 2 \vert M ( {t}_{k} ) ) dSvO 2$$

FIG. 8G illustrate the evaluation of the state of Acidosis based on the ISV of pH of arterial blood. As a non-limiting example the probability of this state is computed as the cumulative distribution of arterial pH [p(pH)] less than a 7.25 threshold. The resulting Clinical Risk can be displayed as an index whose instantaneous time value is given by:

$$AC Index = P ( pH < 7.25 \vert M ( {t}_{k} ) ) = {\int }_{- ∞}^{7.25} P ( pH \vert M ( {t}_{k} ) ) dpH$$

FIG. 9 illustrates a non-limiting example of a definition of a patient state that may be employed by the clinical trajectory interpreter module 123. Specifically, it assumes that the function P(S|ISV₁, ISV₂, . . . , ISVₙ) may be defined by partitioning the domain spanned by the internal state variables ISV₁, ISV₂, . . . , ISVₙ. The particular example assumes that the patient physiology is described by two internal state variables: Pulmonary Vascular Resistance (PVR) and Cardiac Output (CO). The particular risks and respective etiologies that may be captured by these two ISVs emanate from the effects of increased pulmonary vascular resistance on the circulation. Specifically, high PVR may cause right-heart failure and consequently reduced cardiac output. Therefore, PVR can be used to define the attributes of Normal PVR and High PVR, and CO to define the attributes of Normal CO and Low CO, by assigning thresholds with the two variables. By combining these attributed, four separate states can be defined: State 1: Low CO, Normal PVR; State 2: Low CO, High PVR; State 3: Normal CO, High PVR; State 4: Normal CO Normal PVR.

FIG. 10 illustrates a non-limiting example of how the clinical trajectory interpreter module 123 may employ the definition of patient states to assign probabilities that the patient may be classified under each of the four possible patient states at a particular point of time. In the example, the clinical trajectory interpreter module 123 takes the joint probability density function of P(Cardiac Output (Tk), Pulmonary Vascular Resistance (Tk)) and integrates it over the regions corresponding to each particular state, which produces P(S1(Tk)), P(S2(Tk)), P(S3(Tk)), and P(S4(Tk)). In this way, the clinical trajectory interpreter module 123 assigns a probability that a particular patient state is ongoing, given the information provided by the physiology observer module 122. Note that if the output of the physiology observer module 122 is not a closed form function 260 but a histogram 280 of particles 270, the clinical interpreter will not perform integration but just calculate the relative fraction of particles 270 within each region.

FIG. 11 illustrates an alternative approach of estimating the probabilities for different patient states. In this alternative approach, to calculate the probabilities P(S1), P(S2), P(S3) and P(S4), the clinical trajectory interpreter module 123 employs the joint probability functions of the ISVs for two consecutive time windows Tₖ and Tₖ₊₁ to calculate a moving window average. Note in the example that the size of the window is doubled for two time instances, which indicates that the window may be of an arbitrary, suitable size. As a result of this moving window averaging, the clinical trajectory interpreter module 123 performs a dynamic analysis of the trajectory of the ISVs. That is, it gives a metric of the probability that the physiology trajectory, as described by the ISVs, may be found in a particular region in a particular time frame. In other words, this probability calculation gives an estimate of the probability that a particular patient state may be ongoing in the chosen time-frame, as opposed to just at a chosen time instance.

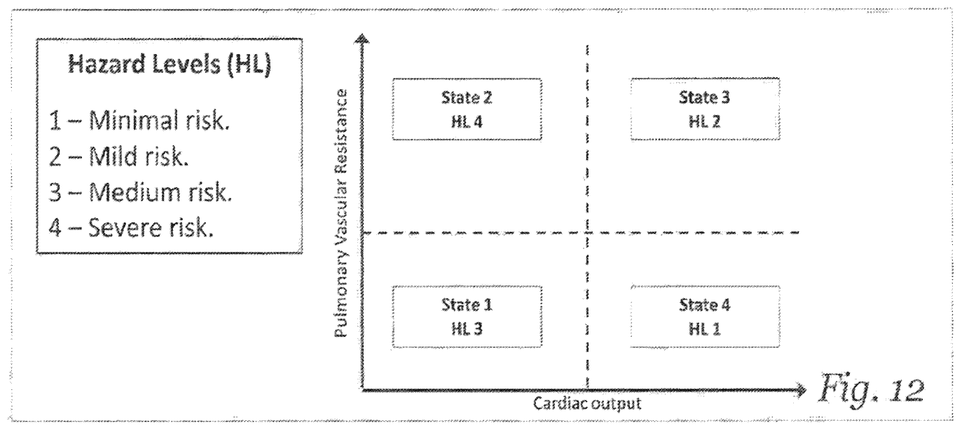

Clinical trajectory interpreter module 123 may also assign hazard levels to each particular state. FIG. 12 illustrates a non-limiting example of a definition of patient states assigned with hazard levels by the clinical trajectory interpreter module 123. The hazard levels may be informed from clinician surveys, reference literature or any other clinical sources. In the particular example, the clinical trajectory interpreter module 123 distinguishes between four different hazard levels: 1—Minimal risk, 2—Mild risk, 3—Medium risk, and 4—Severe risk. The combination of the probability of a patient state and its hazard level will be referred from hereon as a “Patient risk.”

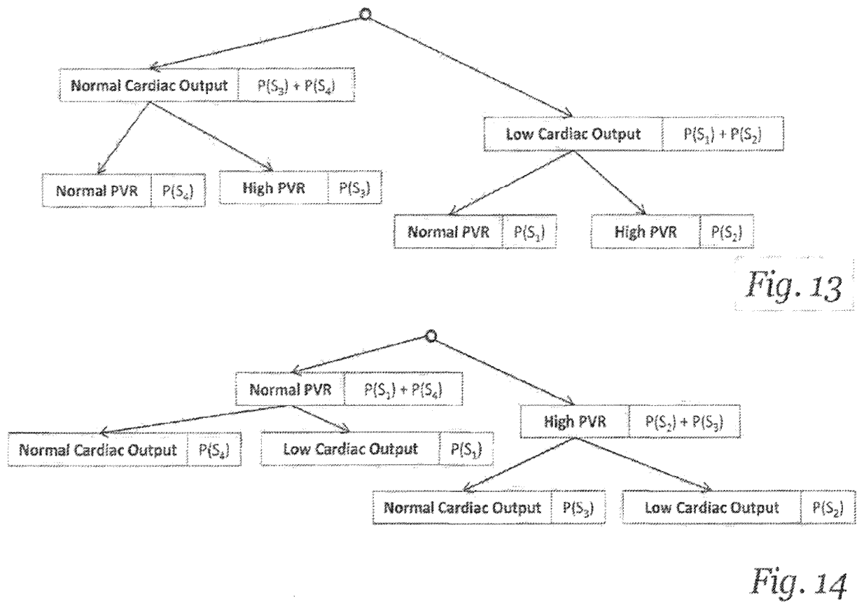

FIG. 13 illustrates how the patient states and their respective probabilities may be organized into tree graphs called etiologies. In particular, the attributes normal and low associated with the cardiac output ISV are the base nodes of the graph. Each of these vertexes has two children associated with the attributes of the pulmonary vascular resistance. This organization leads to each patient state being a leaf (end vertex) on the tree. This particular tree will be referred to as an etiology tree. The etiology tree may be further employed by the visualization and user interaction module 124 to provide a layered view of the various patient risks as further described herein.

FIG. 14 illustrates that the etiology tree may not be unique for a given set of patient states and physiologic variables. Specifically, FIG. 14 provides an alternative etiology tree for the example from FIG. 13. The root of the alternative etiology tree starts from the attributes associated with the pulmonary vascular resistance, instead of the attributes associated with cardiac output. It can be appreciated that different rules may be employed for generating the trees depending on various factors and the context of use. For example, one etiology tree may be preferred against another realization in different clinical situations or depending on the preference of the users. Moreover, the tree may dynamically change as the risks change and the clinical situation evolves.

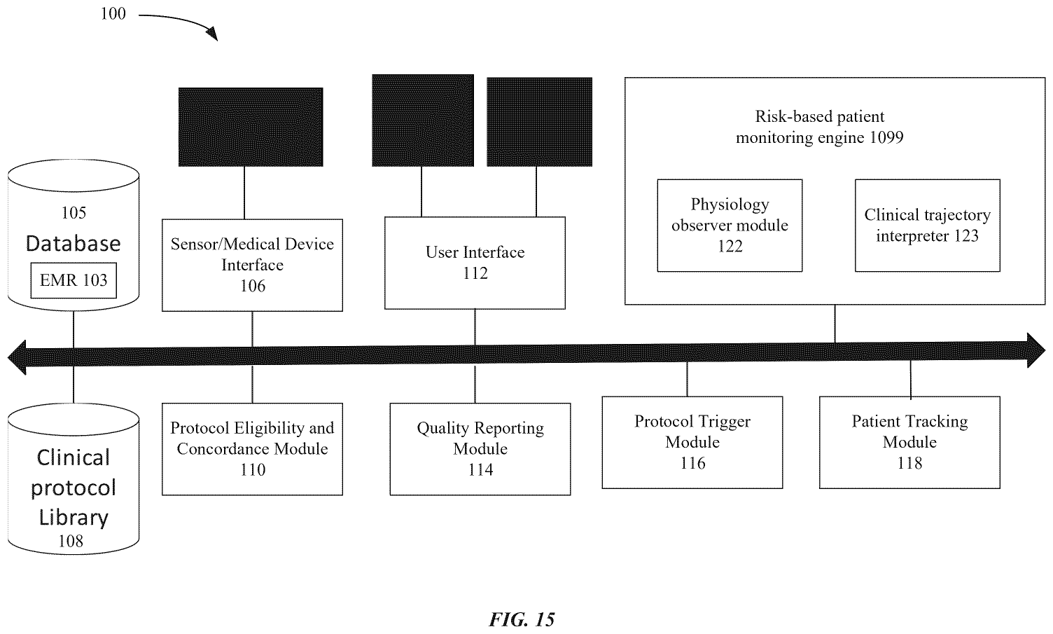

FIG. 15 schematically shows details of an embodiment of system 100 in accordance with illustrative embodiments. The system 100 includes a sensor/medical device interface 106 configured to communicate with the one or more sensors 102 and/or one or more medical devices 104. For convenience, illustrative embodiments may refer to receiving data from one or more sensors and/or one or more medical devices generally as receiving data from the sensors 102. However, it should be understood that discussion of receiving data from the sensors 102 is intended to also include receiving data from the medical devices 104.

To reiterate, the interface 106 receives/streams real-time patient data from the sensors 102. The interface 106 may receive data from a variety of sensors 102, such as blood oximeter, a blood pressure measurement device, a pulse measurement device, a glucose measuring device, one or more analyte measuring devices, an electrocardiogram recording device, amongst others. In some embodiments, the interface 106 simultaneously communicates with a plurality of sensors 102 and/or medical devices. Accordingly, the interface 106 may aggregate and/or compile the various received patient data.

The system 100 includes a database 105 having access to the patient EMR 103, as described previously. The database 105 may also communicate with the sensor interface 106 to store the real-time data as it is received via the interface 106. The system 100 also includes a medical protocol library 108 containing information relating to a plurality of medical protocols. As an example, the medical protocol may include an extubation readiness trial protocol, ventilation vasoactive weaning protocol, sepsis management protocol, enhanced recovery after surgery (ERAS) protocols, and/or other hospital protocols. The information in the library 108 includes eligibility rules and compliance rules for each of the protocols, which are discussed in more detail further below. The library 108 may also include specific subscription rules and/or notification rules for each protocol, which are discussed in more detail further below.

A protocol eligibility and compliance module 110 is configured to determine patient eligibility for at least one of the medical protocols as a function of the received patient data. Additionally, the eligibility and compliance module 110 may access historic data and/or the EMR 103 from the database 105 to determine patient eligibility. To that end, the eligibility and compliance module 110 may communicate with, among other things, the interface 106, the database 105, and the library 108.

Generally, patient eligibility depends on one or more measurements of patient data meeting a threshold status according to the requirements of the protocol stored in the library 108 (e.g., a certain FiO2 and/or BPM measurement). When the patient 101 is eligible for a protocol, the eligibility module 110 triggers an indication that the patient 101 is eligible. In some embodiments, the eligibility module 110 sounds an alert and/or communicates with a user interface 112 to visually indicate the patient is eligible. In some embodiments, the user interface also communicates the amount of time the patient has been eligible. Thus, illustrative embodiments advantageously provide real-time updates as to patient eligibility status. The real-time data collection and eligibility updates contrast to prior art methods that rely on medical practitioners checking, collecting, aggregating, and analyzing a plurality of static patient parameters to determine protocol eligibility.

The determination of eligibility is based on a collection of measurement thresholds, events, and/or other binary variables. For example, eligibility rules for a vasoactive weaning protocol may include that the patients are under cardiac service (determined by the hospital ADT stream or EMR), are on medication drip (another event), have IDO2 index less than a threshold value, and have blood pressure greater than another threshold value.

The system 100 may include a quality reporting module 114 that tracks the time elapsed from patient eligibility until a user acknowledges patient eligibility. Alternatively, or additionally, the quality reporting module 114 may track the time elapsed from patient eligibility until a medical practitioner begins the course of action associated with the eligible protocol for the patient 101. By tracking this information, medical practitioner performance may be objectively measured (e.g., which practitioners are the fastest responders and what are the impacts of fast response time on clinical outcomes).

The system 100 may also include a protocol trigger module 116 that instructs the eligibility and compliance module 110 to begin tracking patient 101 compliance data. The protocol trigger module 116 is configured to receive information regarding the initiation of a course of action in the protocol 120. In various embodiments, the medical practitioner may initiate the course of action based on the feedback from the eligibility module 110 and inform the protocol trigger module 116 (e.g., through the user interface) of the course of action initiation. In some embodiments, the protocol trigger module 116 is configured to automatically initiate the course of action by communicating with a treatment device 104 (e.g., after a medical practitioner approves the initiation via the user interface). In other embodiments, the protocol trigger module 116 can automatically detect that the practitioner has initiated the course of action and initiate tracking concordance data. For example, in the ERT protocol, when the system 100 detects that the ventilator mode has been changed, and this is used to detect the start of an ERT protocol and triggers dynamic concordance tracking for the protocol.

After the course of action for the protocol is initiated, the concordance module 110 may continue to communicate with the same, different, or additional sensors 102 to determine patient 101 compliance with the protocol. The compliance module 110 may determine the compliance of the patient 101 with the medical protocol as a function of newly received patient data and the compliance rules in the library 108. Compliance is generally determined as a calculation of whether the patient data meets acceptable levels set by the library 108 for the particular protocol (e.g., the patient variable is compared to the level or range required by the library 108). Additionally, the compliance module 110 may visually indicate (e.g., via the user interface 112) the real-time patient dynamic concordance status. The concordance module 110 may send the dynamic concordance rate to the user interface 112, which displays the dynamic concordance rate. Accordingly, the attention of the medical practitioner may be drawn to the patient in need of action.

The quality reporting module 114 may track a dynamic concordance rate as a percentage of time over which the patient 101 is compliant with the medical protocol and/or the patient eligibility time. Additionally, the quality reporting module 114 may retrospectively look at patient concordance rate and/or eligibility time to see how well a particular patient and/or patients of a particular medical practitioner have performed relative to a patient population. Additionally, the reporting module 114 may be configured to display the received patient data and to mark non-compliant portions of the received data. Thus, the quality reporting module 114 allows medical facilities and/or staff to evaluate how well patients were managed retrospectively. For example, if eligibility times are very high, medical practitioners become aware of it. This assists with hospital policy formation and also with ensuring prompt action by the medical staff.

The system of FIG. 15 may include a risk-based monitoring engine 1099 (also referred to as “risk engine 1099”) configured to receive data from bedside monitors 102, electronic medical records 103, treatment devices 104, and any other information that may be deemed relevant to make an informed assessment regarding the patient's clinical risks, and any combination thereof of the preceding elements.

In some embodiments, components of the system may be separated into different components. For example, the protocol eligibility and compliance module may be split into two different modules, e.g., a protocol eligibility module and a protocol compliance module. Additionally, various components, such as the sensor/medical device interface 106 of FIG. 15, may be distributed across a plurality of different machines—not necessarily within the same housing or chassis.

Additionally, in some embodiments, components shown as separate (such as the eligibility and compliance module 110 and the quality reporting module 114 in FIG. 15) may be replaced by a single component. Illustrative embodiments may include additional modules not explicitly shown here. Furthermore, certain components and sub-components in FIG. 15 are optional. For example, some embodiments may not include the protocol trigger module 116.

It should be reiterated that the representation of FIG. 15 is a significantly simplified representation of the system 100. Those skilled in the art should understand that such a device may have other physical and functional components, such as central processing units, other packet processing modules, and short-term memory. Indeed, the system 100, in various embodiments, includes one or more of the following: a processor, a memory coupled to the processor, and a network interface configured to enable the system to communicate with other devices over a network. In addition, the system may include a risk-based monitoring application 1020 that may include computer-executable instructions, which when executed by the processor 111, cause the system to be able to afford risk-based monitoring of the patients, such as the patient 101. Accordingly, this discussion is not intended to suggest that FIG. 15 represents all of the elements of the system 100.

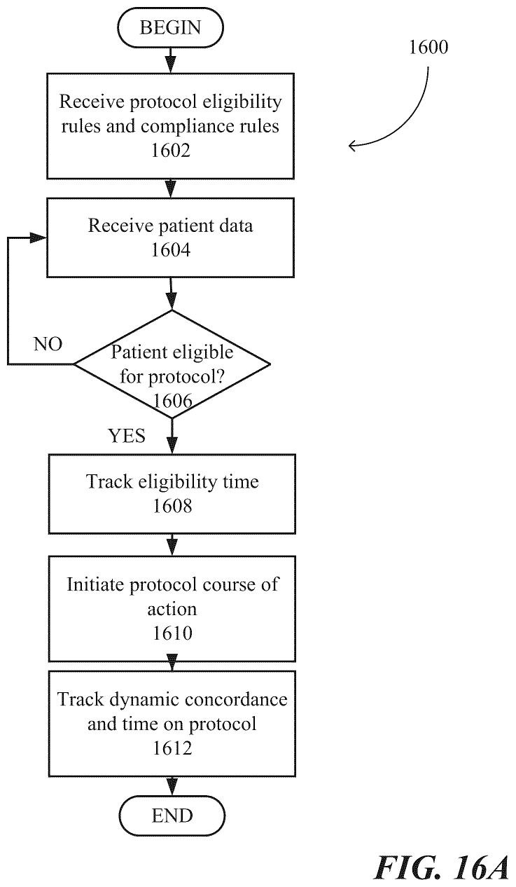

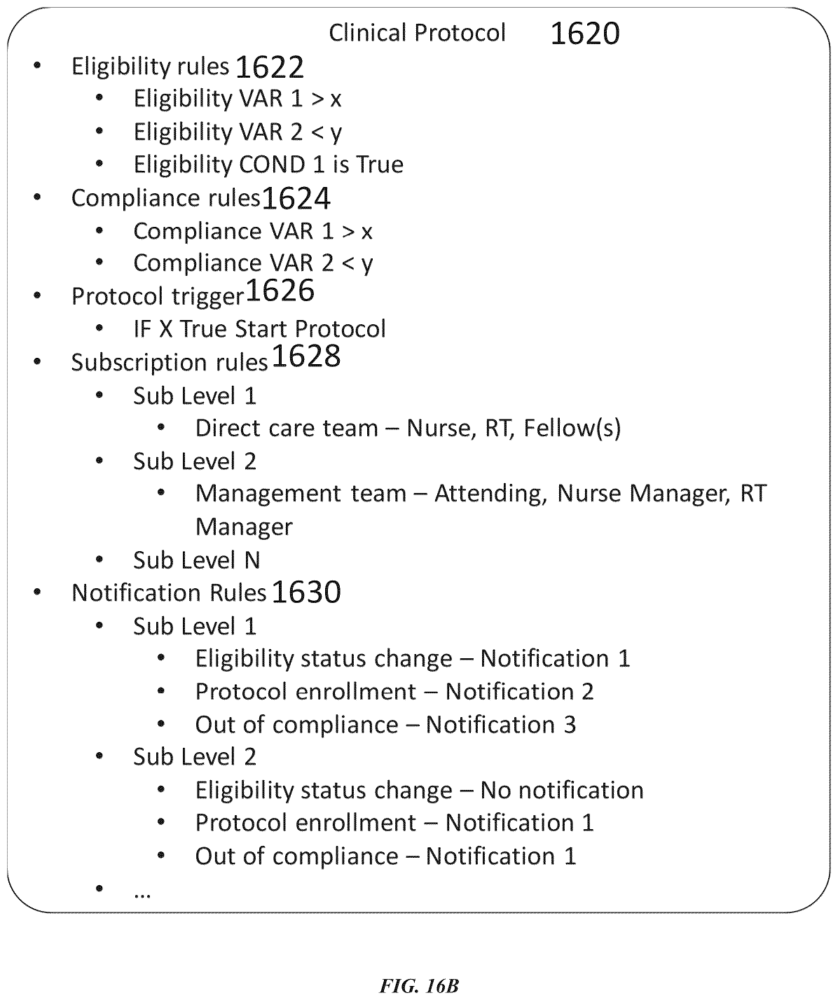

FIG. 16A shows a process 1600 of determining that the patient 101 is eligible for the medical protocol. It should be noted that this process can be a simplified version of a more complex process that may normally be used. A s such, the process may have additional steps that are not discussed. In addition, some steps may be optional, performed in a different order, or in parallel with each other. Accordingly, discussion of this process is illustrative and not intended to limit various embodiments. Finally, although this process is discussed with regard to entering a single patient in a medical protocol, the process of FIG. 16A can be expanded to cover processes for entering a plurality of patients in one or more medical protocols at the same time. Accordingly, the process 1600 is merely exemplary of one process in accordance with illustrative embodiments. Those skilled in the art therefore can modify the process as appropriate.

The process begins at step 1602, which receives eligibility rules and compliance rules for one or more protocols. In illustrative embodiments, the eligibility rules and compliance rules are received from the medical protocol library 108. As described previously, the protocol library 108 contains information relating to a plurality of medical protocols.

FIG. 16B schematically shows a generic example of a medical protocol 1620 having a variety of eligibility rules 1622, compliance rules 1624, a protocol trigger 1626, subscription rules 1628, and notification rules 1630 in accordance with illustrative embodiments. Although the eligibility rules 1622, the compliance rules 1624, the protocol trigger 1626, subscription rules 1628, and the notification rules 1630 are shown as being part of a single protocol 1620, in some embodiments, the protocol eligibility rules 1622, the compliance rules 1624, the protocol trigger 1626, subscription rules 1628, and/or the notification rules 1630 may be received at different times within the process of FIG. 16A. Additionally, in some embodiments, the medical protocol 1620 may not include subscription rules 1628, the notification rules 1630, the compliance rules 1624 and/or the protocol triggers 1626.

The eligibility rules 1622 may include one or more conditions that must be satisfied (e.g., true or false) for the patient 101 to be eligible for the protocol 1620. The eligibility module 110 may compare data in the database 105 and/or EMR 103 with the rules 1622 to determine whether the rules 1622 are met. Additionally, or alternatively, the eligibility rules 1622 may include one or more parametric conditions that must be satisfied (e.g., a certain variable is greater than a particular value). The eligibility module 110 may receive patient data 106 from the interface 106 to determine whether the patient 101 is eligible (e.g., the rules 1622 are satisfied).

In a similar manner, the compliance rules 1624 may include a variety of conditions that must be satisfied (e.g., true or false) for the patient 101 to be in compliance with the protocol 1620. The compliance module 110 may compare data in the database 105 and/or EMR 103 with the rules 1622 to determine whether the rules 1622 are met. Additionally, or alternatively, the compliance rules 1624 may include a variety of parametric conditions that must be satisfied (e.g., a certain variable is greater than a particular value). The compliance module 110 may receive patient data 106 from the interface 106 to determine whether the patient is in compliance with the protocol 1620 (e.g., the rules 1624 are satisfied).

Furthermore, in some embodiments the protocol 1620 may define a protocol trigger 1626 that includes one or more conditions that automatically trigger the course of action to begin when satisfied. In some embodiments, the conditions may include a dynamic concordance rate of the given protocol or of a parent protocol. The protocol trigger module 116 may detect when these conditions are satisfied, and may communicate with and/or control one or more medical devices 104 to begin the course of action. Additionally, or alternatively, the protocol trigger module 116 may receive an indication (e.g., from a medical device 104 or from a medical practitioner through the user interface) that instructs the protocol trigger module 116 to activate the compliance module 110. The compliance module 110 then begins to track dynamic concordance.

In various embodiments, the protocol 1620 may further include subscription rules 1628 and notification rules 1630. The subscription rules 1628 assign different subscription levels to different medical practitioners. For example, the direct care team may be subscription level 1, whereas the management team by may be subscription level 2. The subscription rules 1628 may be used in the notification rules 1630. Accordingly, different subscriptions may receive different notifications and/or notifications for different reasons.

The process then proceeds to step 1604, which receives patient data directly or indirectly. The patient data may be received directly (e.g., streamed) in real-time from the sensors 102 and/or medical devices. The patient data may include internal state variables (or “ISV”), which are parameters of the patient's physiology that is physiologically relevant to treatment and/or a condition of a patient. ISVs may be directly observable with noise (as a non-limiting example, heart rate is a directly observable ISV), hidden (as a non-limiting example, oxygen delivery (DO2) defined as the flow of blood saturated oxygen through the aorta cannot be directly measured and is thus hidden), or measured intermittently (as a non-limiting example, hemoglobin concentration as measured from Complete Blood Count tests is an intermittently observable ISV). Other examples of ISVs include, without limitation, Pulmonary Vascular Resistance (PVR); Cardiac Output (CO); hemoglobin, and rate of hemoglobin production/loss.

Additionally, in some embodiments, the relevant patient data may be indirectly received (e.g., may not be directly observable/measurable). A hidden Internal State Variable, means an ISV that is not directly measured by the sensor 102 coupled to the patient 101. Some hidden ISVs cannot be directly measured by the sensor. In some embodiments, the module may receive, in addition to or instead of sensor data, data representing a risk that the patient is suffering a specific adverse medical condition as indicated by the probability of the hidden internal state variable being in a particular state. Some hidden ISVs require, for example, laboratory analysis of a sample (e.g., blood) taken from the patient. Additional details regarding hidden internal state variables are described in U.S. patent application Ser. No. 17/033,591, issued as U.S. Pat. No. 11,676,730, each of which is incorporated herein by reference in its entirety.

Among other things, the received patient data may include: expired CO2, end-tidal CO2 measurement (EtCO2), arterial CO2, minute ventilation, ventilator mode, drug infusion rate, respiratory rate, PaCO2 arterial blood gases, Hb Hemoglobin, HR Heart Rate, SpvO2 Pulmonary Venous Oxygen Saturation, SaO2 Arterial Oxygen Saturation, SvO2 Systemic Venous Oxygen Saturation, SpO2 Pulmonary Venous Oxygen Saturation, Mean Arterial Blood Pressure, Central Venous Pressure, Left Atrial Pressure, Right Atrial Pressure, patient weight, patient age, patient height, and/or patient medical history.

Returning to FIG. 16A, the process proceeds to step 1606, which determines whether the patient 101 is eligible for the protocol 1620. The protocol eligibility and compliance module 110 compares the various patient data with the eligibility rules 1622 to determine whether the patient 101 is eligible for the protocol 1620.

### Nested Protocols

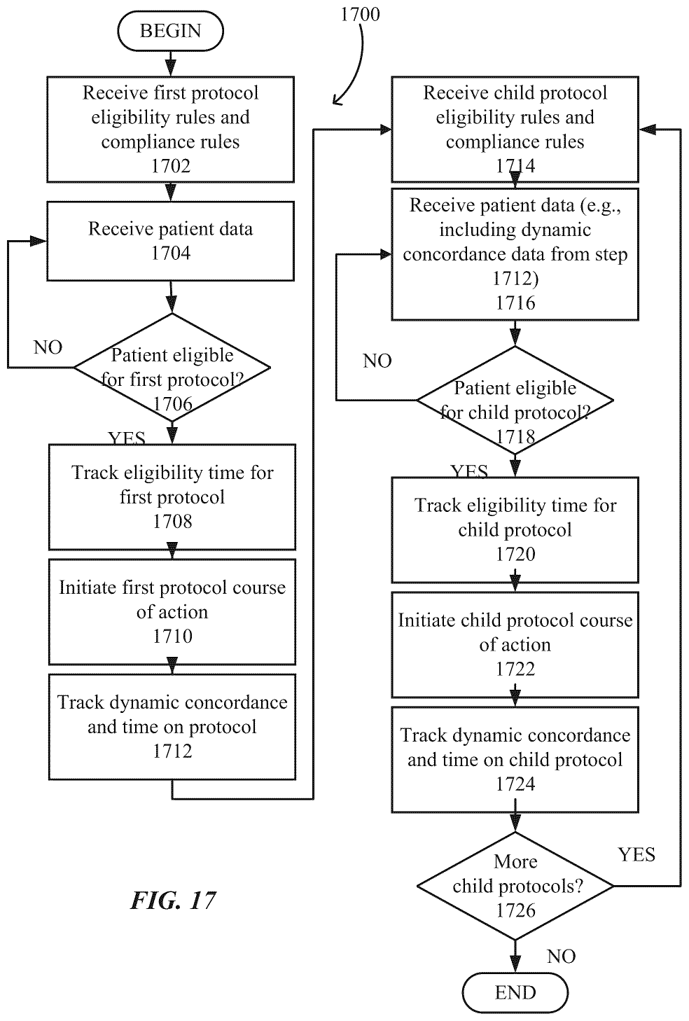

FIG. 17 shows a process 1700 of nesting protocols in accordance with illustrative embodiments. It should be noted that this process can be a simplified version of a more complex process that may normally be used. As such, the process may have additional steps that are not discussed. In addition, some steps may be optional, performed in a different order, or in parallel with each other. Accordingly, discussion of this process is illustrative and not intended to limit various embodiments. Finally, although this process is discussed with regard to entering a single patient in a medical protocol, the process of FIG. 17 can be expanded to cover processes for entering a plurality of patients in one or more medical protocols at the same time. Accordingly, the process 1700 is merely exemplary of one process in accordance with illustrative embodiments. Those skilled in the art therefore can modify the process as appropriate.

Steps 1702-1712 are substantially the same as steps 1602-1612 of FIG. 16A, respectively, and therefore, discussion of these steps is not again repeated in great detail. Steps 1702-1712 generally describe a process of determining whether a patient is eligible for a first protocol, and tracking the dynamic concordance rate of the patient after a course of action of the protocol begins.

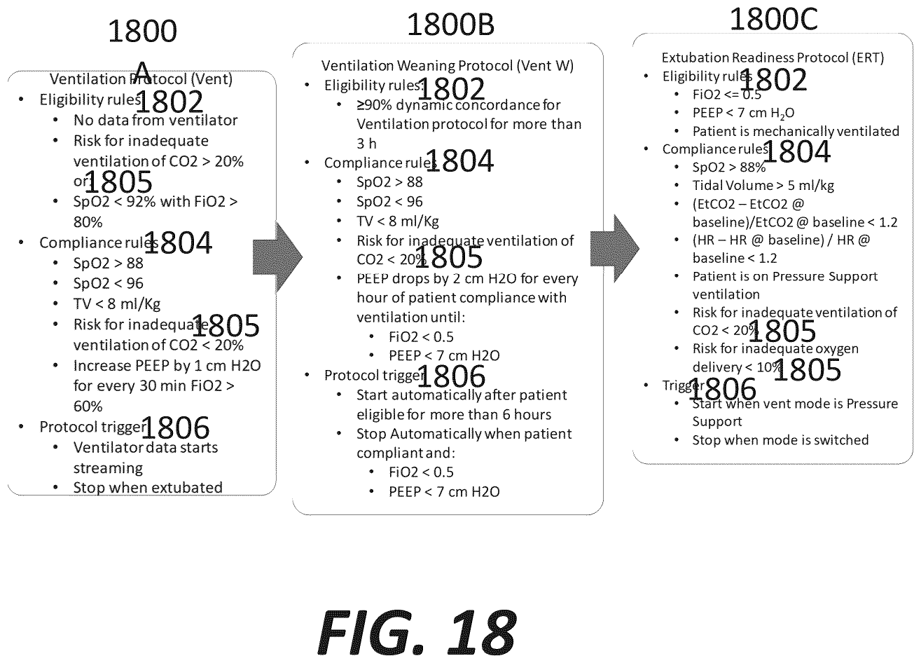

FIG. 18 schematically shows an example of nested medical protocols having a variety of eligibility rules, compliance rules, and a protocol trigger in accordance with illustrative embodiments. As shown in the example of FIG. 18, the eligibility rules 182 of the ventilation weaning protocol 180B are dependent upon a particular outcome from the ventilation protocol 180A. For the sake of discussion, the dependent protocol 180B may be referred to as the child protocol 180B. Whereas the protocol 180A, upon which the child protocol 180B depends, may be referred to as a parent protocol 180A. Despite the use of this nomenclature, it should be understood that a child protocol 180B may itself in turn be a parent protocol for a different protocol (e.g., a third protocol 180C). Furthermore, although not shown in this example, a child protocol 180B may have multiple parent protocols 180A upon which it depends. In a similar manner, a parent protocol may have multiple child protocols, with each child triggered by different conditions associated with the parent protocol (e.g., different dynamic concordance rates may trigger different child protocols).

In a manner similar to the previously described protocol 1620 of FIG. 16B, each of the protocols 180A-180C in FIG. 18 has a respective set of eligibility rules 182, a course of action or trigger 186, and compliance rules 184. In the process of FIG. 17, steps 1702-1712 may relate to, for example, the first protocol 180A.

At step 1710, the course of action (e.g., ventilation) begins when the eligibility rules 182 of the first protocol 180A are satisfied. After the course of action begins, the dynamic concordance rate for the various compliance rules 184 is tracked at step 1712.

Step 1714 receives the eligibility rules 182 and the compliance rules 184 for the second protocol 180B. Although step 1714 is shown as occurring after step 1712, in practice, this step may occur earlier in the process 1100 (e.g., simultaneously with 1702).

At step 1716, the process receives patient data, which includes the dynamic concordance rate of the first protocol 180A (also referred to as the parent protocol). At step 1718, the process 1100 determines if the patient is eligible for the second protocol. Step 1718 compares the received data from step 1716 with the eligibility rules 1822 for the protocol 180B. Among other things, the eligibility rules 182 for the child protocol 180B (the nested protocol) may include the dynamic concordance rate from another protocol (e.g., the parent protocol 180A). In illustrative embodiments, the system 100 enables the use of nested protocols by keeping track of the status of each protocol 180A-180C and continuously calculating dynamic concordance rate.

In the example of FIG. 18, the second protocol 1802 requires a 90% or greater dynamic concordance for more than 3 hours for the ventilation protocol 1800A. The system 100 tracks all of the relevant data relating to the compliance 1804 of the first protocol 1800A, and calculates the dynamic concordance rate. Although the eligibility rules show a reliance only on a single parent protocol (i.e., the ventilation protocol 1800A), it should be understood that eligibility rules 1802 for a protocol may include dynamic concordance rates (or other variables) from multiple other protocols. Thus, a single child protocol 1800B may have multiple parent protocols 1800A.

Additionally, illustrative embodiments enable the use of various nested eligibility rules 1802 and/or compliance rules 1804. For example, rules 1802 and 1804 may rely on an overall dynamic concordance rate for one or more parent protocols 1800A, a dynamic concordance rate for a most recent period of time (e.g., 90% or greater for the most recent 3 hours) for the one or more parent protocols 1800A, a total minimum time above threshold dynamic concordance rate (e.g., 2 hours above threshold, 1 hour below threshold, and 1 hour above threshold), and/or a dynamic concordance rate that is a function of a plurality of parent protocols 1800A (average dynamic concordance rate for plurality of parent protocols 1800A is above a given threshold). Various embodiments may include Boolean operators in the eligibility rules 1802 and/or the compliance rules 1804 (e.g., “and,” “or,” “not”).

Returning to FIG. 17, after determining that the patient 101 is eligible for the second protocol 1800B, the process 1100 proceeds to step 1720, which begins tracking eligibility time for the second protocol 1800B. In a manner similar to step 1708, step 1718 provides a quantitative measurement of hospital staff performance, and also provides the medical practitioner with information which may be used to prioritize the treatment of patients in the unit (and/or the medical practitioner's attention to a particular patient).

Although not explicitly shown, throughout the process of FIG. 17 notifications may be sent to medical staff in accordance with subscription rules 1808 and the notification rules 130 of the protocol. For example, notifications of patient eligibility for the second protocol are sent to responsible hospital staff. Illustrative embodiments advantageously reduce unnecessary pending eligible time to initiation for patients 101 eligible for the second protocol. Furthermore, illustrative embodiments advantageously enable the user of dynamic concordance rates from the first protocol 1800A as an eligibility requirement for the second protocol 1800B.

The inventor believes that both of these features (i.e., reduced pending eligibility time, and focus on concordance rate based outcomes) lead to enhanced patient outcomes. The reason is that they enable the medical practitioner to provide care, which keeps the patient on the optimal progression path thus reducing time in the ICU and recovery. In the example from FIG. 17, the patient is intubated earlier by a timely notification that the patient is eligible for ventilation. The ventilation dynamic concordance rate enables the medical practitioner to longitudinally track whether the patient is receiving optimal ventilation and oxygenation, and to intervene in a timely manner if necessary. Expedited intervention reduces the risk for end-organ failure. After the patient is stabilized, the system 100 prompts the practitioner to start weaning, which effectively mitigates the risk of the patient 101 spending too much time on ventilation and experiencing ventilator associated lung injury. This further impacts the timeliness of downstream child protocols, such as the ERT protocol. The system 100 allows the clinician to know how much time the patient 101 may have been eligible for a particular protocol (e.g. the extubation trial), thus urging them to start the protocol. After the trial is completed, the system 100 provides the dynamic concordance rate to the whole clinical team during rounds or other patient care planning activity so they may timely make a decision for initiation of another protocol and/or course of action (e.g., extubation).

At step 1722, the course of action of the second protocol 1800B begins (i.e., the patient 101 is enrolled in the protocol). After the second protocol 1800B begins, dynamic concordance of the patient is tracked. The process then proceeds to step 1726, which asks if there are more protocols. If the answer is yes, then more patient data is received. Returning to the example of FIG. 18, a third protocol 1800C has eligibility rules that relate an earlier protocol 1800B. Accordingly, steps 1714-1724 are repeated for the third protocol 1800C. This process may be repeated for as many protocols as necessary. Eventually, when there are no additional nested protocols, the process comes to an end.

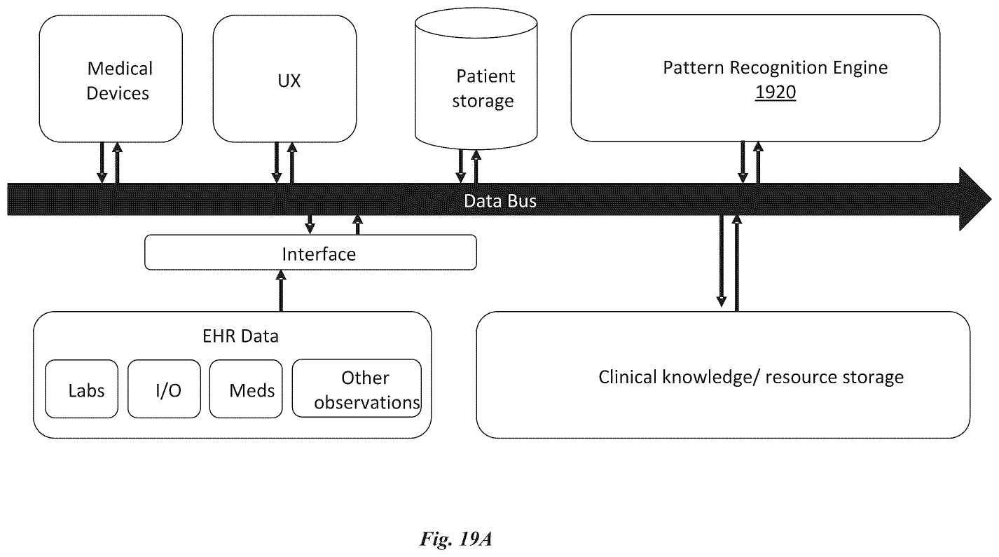

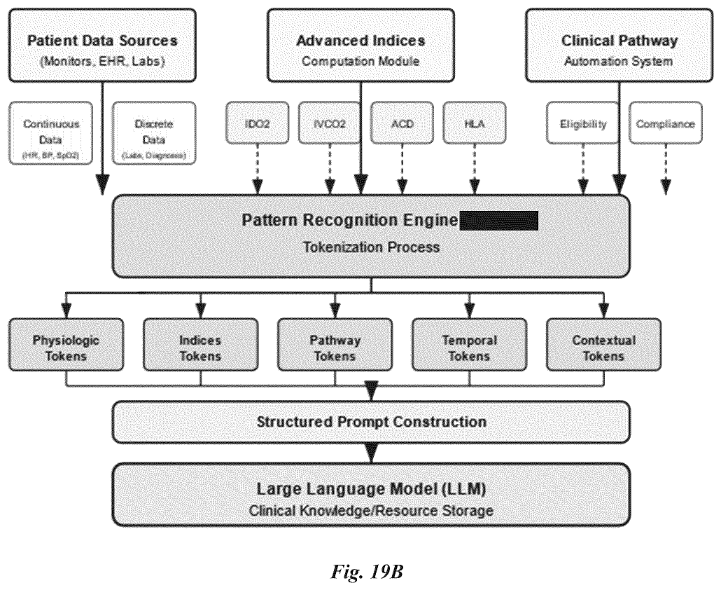

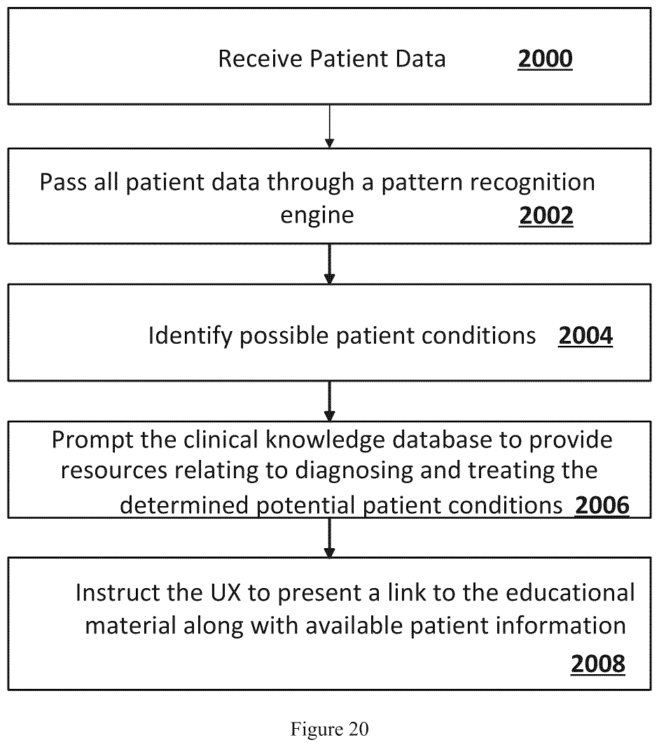

FIG. 19A schematically shows a knowledge platform in accordance with illustrative embodiments. Each of these components is operatively connected by any conventional interconnect mechanism. FIG. 19A simply shows a bus communicating each the components. Those skilled in the art should understand that this generalized representation can be modified to include other conventional direct or indirect connections. Accordingly, discussion of a bus is not intended to limit various embodiments.

Indeed, it should be noted that FIG. 19A only schematically shows each of these components. Those skilled in the art should understand that each of these components can be implemented in a variety of conventional manners, such as by using hardware, software, or a combination of hardware and software, across one or more other functional components. For example, the pattern recognition engine (discussed below) may be implemented using a plurality of microprocessors executing firmware. As another example, the pattern recognition engine may be implemented using one or more application specific integrated circuits (i.e., “ASICs”) and related software, or a combination of ASICs, discrete electronic components (e.g., transistors), and microprocessors. Accordingly, the representation of the pattern recognition engine and other components in a single box of FIG. 19A is for simplicity purposes only. In fact, in some embodiments, the pattern recognition engine of FIG. 19A is distributed across a plurality of different machines—not necessarily within the same housing or chassis.

It should be reiterated that the representation of FIG. 19A is a simplified representation of an actual knowledge platform Those skilled in the art should understand that such a device has many other physical and functional components, such as central processing units, other packet processing modules, and short-term memory. Accordingly, this discussion is in no way intended to suggest that FIG. 19A represents all of the elements of a knowledge platform or the like.

As shown, the knowledge platform is equipped with a graphical user interface (UX) designed to enhance accessibility for users, such as clinicians. The UX simplifies the process of navigating the system, allowing healthcare professionals to efficiently access and interact with the necessary tools and information. By focusing on usability, the platform ensures that clinicians can focus more on patient care rather than struggling with complex software navigation. The intuitive design of the UX helps in reducing the learning curve and increases the overall effectiveness of the system in a clinical setting.

The user then may access a variety of different functions via the UX. To that end, the knowledge system has patient storage (e.g., a database) containing patient information, clinical knowledge/resource storage containing clinical educational material, and electronic health record storage (“EHR Data”), communicating with the bus via an interface (e.g., an application programming interface), containing more patient information typically found in an electronic health record.

As known by those in the art, an electronic health record (EHR) is a digital version of a patient's paper chart and is a critical component of health information technology. It systematically collects a patient's health information and medical history in a format that can be shared across different healthcare settings. EHRs typically contain comprehensive data about a patient, including personal identification like name and date of birth, contact information, and insurance details. They also often store medical data such as diagnoses, medications, treatment plans, immunization dates, allergies, radiology images, and laboratory and test results. Information about past medical history, including surgeries and other procedures, family medical histories, and physician notes and observations, often also are parts of an EHR.

The use of EHRs enhances the ability of healthcare providers to make well-informed treatment decisions by providing access to the complete health records of a patient. For example, a physician can quickly review a patient's history of hypertension, note previous medications and their effectiveness, and adjust a treatment plan accordingly. The system also aids in ensuring patient safety through checks for potential harmful drug interactions, facilitates prescriptions electronically, and enables direct lab test ordering and receiving results. Overall, EHRs not only streamline administrative processes but also improve the accuracy of diagnoses and health outcomes. They are a tool in modern healthcare that supports the continuity of care, enhances patient engagement, and optimizes the efficiency of healthcare services.

Each of the three storage devices generally shown as blocks in FIG. 19A offers flexible deployment configurations to suit various application needs, including distributed, centralized, or coordinated setups. In a distributed configuration, data is spread across multiple locations to enhance accessibility and resilience against failures. Conversely, a centralized approach consolidates data into a single location, simplifying management and data retrieval but may increase vulnerability to single-point failures. The coordinated configuration involves synchronizing data across multiple sites, combining elements of both centralized and distributed systems to optimize data consistency and availability. This adaptability allows developers and system architects to tailor the storage architecture precisely to the specific requirements and challenges of the application in question.

The knowledge platform also has a pattern recognition engine that uses information from the various noted storage devices, as well as real-time or current information from medical devices interacting with the patient (e.g., blood pressure cuff, oxygen sensor, and other medical devices commonly used in a hospital or other medical context) to develop a preliminary assessment of the patient. Using this assessment, the pattern recognition engine may preliminarily determine that the patient may have a certain set of conditions (e.g., one or more conditions). Based on that preliminary (but not necessarily a final diagnosis), the pattern recognition engine may direct a clinician or other user to resources to assist in diagnosing and treating the condition.

FIG. 19B schematically illustrates an embodiment of a system operation. In illustrative embodiments, the pattern recognition engine 1920 represents an advanced process for converting both continuous and discrete patient physiologic data into standardized tokens that can be processed by an artificial intelligence system. This tokenization approach significantly enhances the system's ability to identify potential patient conditions and direct users to appropriate resources.

The pattern recognition engine (1920) implements a sophisticated tokenization methodology that transforms various types of patient data into a standardized format suitable for advanced analysis. The tokenization process includes the following steps.

1. Data Acquisition: The system collects continuous physiologic data (e.g., heart rate, blood pressure, pulse oximetry, respiratory rate) and discrete data points (e.g., laboratory results, medication administrations, clinical observations) from multiple sources including bedside monitors, EHR systems, and other medical devices. Such data acquisition may be performed by an embodiment of a system 100, as described herein.

2. Feature Extraction: Key features are extracted from the normalized data, including for example one or more of:
a. Trend directions and rates of change
b. Variability metrics
c. Relationship patterns between different parameters
d. Deviation from patient-specific or population-based norms

Temporal patterns such as cyclical variations or sudden changes.

3. Tokenization The extracted features are converted into discrete tokens that represent clinically meaningful patterns or states. These tokens serve as a standardized language that bridges the gap between raw physiologic data and clinical interpretation.

### Integration of Advanced Clinical Indices

Illustrative embodiments of the tokenization process include the incorporation of advanced clinical indices. Such clinical indices may be produced by one or more embodiments of system 100 as described herein. In some embodiments, the clinical idiocies include one or more of the IDO2 Index, IVCO2 Index, ACD Index, and HLA Index—as high-level tokens that represent complex physiological states:

IDO2 Index Tokenization: The Inadequate Delivery of Oxygen Index is computed through probabilistic modeling of mixed venous oxygen saturation (SvO2) based on multiple physiologic measures. The system tokenizes not only the numerical value of the IDO2 Index but also its rate of change, duration of elevation, and relationship to other parameters, creating a multidimensional token that represents the patient's oxygen delivery status.

IVCO2 Index Tokenization: The Inadequate Ventilation of Carbon Dioxide Index is similarly tokenized, capturing the likelihood of inadequate carbon dioxide ventilation. The token encodes the index value along with contextual information such as the ventilator settings, patient's respiratory efforts, and temporal progression.

ACD Index Tokenization: TheAcidemia Index is processed into tokens that represent notjust the probability of arterial pH below a threshold, but also the likely etiology (respiratory vs. metabolic) based on additional parameters. This allows the system to create more nuanced tokens that carry information about the underlying physiological processes.

HLA Index Tokenization: The Hyperlactatemia Index tokenization captures the probability of elevated lactate levels, with additional encoded information about the likely cause (e.g., tissue hypoxia, medication effect, liver dysfunction) based on the pattern of other physiologic variables.

These indices are particularly valuable as tokens because they already represent a sophisticated fusion of multiple data points into clinically meaningful constructs. By incorporating them into the tokenization process, the system leverages their established clinical validity while enhancing their utility through integration with other patient data.

### Clinical Pathway Automation Integration

In some embodiments, the tokenization process further incorporates elements from the Clinical Pathway Automation system, enhancing the tokens with protocol-specific context:

Eligibility Tokens: The system creates tokens that represent a patient's eligibility status for specific clinical protocols, encoding not just binary eligibility but the degree of match and specific criteria that are or are not met.

Compliance Tokens: For patients enrolled in clinical pathways, the system generates tokens that represent their compliance status with protocol requirements, including duration of compliance, specific parameters that are out of range, and trajectory of compliance over time.

Temporal Eligibility Tokens: The system generates tokens that represent how long a patient has been eligible for a protocol, capturing the urgency of intervention based on prolonged eligibility without protocol initiation.

Dynamic Concordance Tokens: The system creates tokens that represent the patient's dynamic concordance rate with protocol requirements, encoding the percentage of time in compliance and trends in compliance status.

By incorporating clinical pathway elements into the tokenization process, the system can generate tokens that provide context about the patient's status within established care protocols, enriching the subsequent analysis.

### Large Language Model Integration

Once patient data has been converted into a comprehensive set of tokens, the system employs a novel approach to clinical decision support:

Prompt Construction: The tokenized patient data is assembled into structured prompts that frame the clinical question in a format optimized for large language model (LLM) interpretation. These prompts combine the tokens representing the patient's physiologic state with contextual information about their clinical history, demographics, and current treatments.

Clinical Knowledge Resource Storage: The system maintains a connection to a specialized large language model that serves as the clinical knowledge/resource storage component. This LLM has been specifically trained on medical literature, clinical guidelines, and expert knowledge to understand the clinical significance of various physiological patterns.

Context-Enhanced Analysis: When prompted with the tokenized patient data, the LLM generates detailed patient insights that include:
Potential diagnoses with probabilistic assessments
Physiological interpretation of observed patterns
Relevant clinical considerations and caveats
Suggested additional assessments or monitoring
References to applicable clinical guidelines or literature
Educational content specific to the identified conditions

Resource Direction: Based on the LLM's analysis, the system identifies and directs users to appropriate educational resources, relevant clinical protocols, similar case studies, or other support materials that may assist in clinical decision-making.

Confidence Assessment: The system provides transparency about the confidence level of its analysis, highlighting areas where additional data would enhance the reliability of the insights.

Clinical Implementation Advantages. This enhanced tokenization and LLM integration approach offers one or more of several advantages over traditional clinical decision support systems:

1. Comprehensive Data Integration: By tokenizing data from multiple sources, including advanced indices and clinical pathway status, the system can provide insights based on a more complete picture of the patient's condition than would be possible through isolated parameter analysis.

2. Temporal Context Preservation: The tokenization process preserves information about the temporal relationships between different parameters, allowing the LLM to consider the sequence and evolution of physiological changes in its analysis.

3. Adaptive Learning: The system can be designed to update its tokenization strategies based on feedback and outcomes, continuously refining the mapping between raw data patterns and clinically meaningful tokens.

4. Explainable Insights: By using a structured tokenization process before engaging the LLM, the system creates an interpretable intermediate representation that enhances the explainability of the resulting insights.

5. Resource Optimization: The directed educational resources are specifically tailored to the patient's condition, providing clinicians with relevant information at the point of care without requiring extensive manual searches.

In summary, the enhanced pattern recognition engine with tokenization and LLM integration represents a significant advancement in the system's ability to support clinical education and decision-making. By converting complex physiological data into standardized tokens and leveraging the capabilities of large language models, the system can provide nuanced clinical insights and direct users to appropriate resources with unprecedented specificity and relevance.

### Implementation Example

FIG. 19B schematically illustrates the enhanced tokenization and LLM integration process of the knowledge platform in accordance with these additional embodiments. As shown, the process includes a data collection module that feeds into the tokenization engine, which generates standardized tokens incorporating the advanced indices and clinical pathway status. These tokens are then formatted into structured prompts that are processed by the clinical knowledge LLM, which generates detailed patient insights and directs users to appropriate educational resources.

FIG. 20 shows an embodiment of a process that the knowledge engine of FIG. 20 may use to direct a user toward a clinical resource. It should be noted that this process is a simplified from a longer process that normally would be used to direct users toward resources. Accordingly, the process of may have many steps that those skilled in the art likely would use. In addition, some of the steps may be performed in a different order than that shown, or at the same time. Those skilled in the art therefore can modify the process as appropriate. Moreover, as noted above and below, many of the structures noted are but one of a wide variety of different structures that may be used. Those skilled in the art can select the appropriate materials and structures depending upon the application and other constraints. Accordingly, discussion of specific structures is not intended to limit all embodiments.

The process begins at step 2000, which receives patient data. That data may be received from any one or more of the sources noted above in FIG. 19A (e.g., the patient storage, medical devices, and/or electronic health record). Next, that information is passed into the pattern recognition engine for analysis (step 2002) to identify possible patient conditions (step 2004).

Specifically, the pattern recognition engine leverages vast amounts of data from the noted data stores (or other other data stores), combined with real-time or recently derived health information from tests and labs to identify potential health conditions in patients. This identification process often begins with the integration and analysis of historical data, such as past medical diagnoses, treatment outcomes, and family medical history, stored within the EHR. By consolidating this data, the pattern recognition engine can establish a comprehensive health profile for each patient, allowing for the detection of patterns or anomalies that may indicate underlying health issues.

In addition to historical data, the platform may regularly incorporate current health information from recent lab tests and diagnostic reports. This real-time data provides immediate insights into a patient's current health status, enabling the platform to identify acute conditions that may require urgent attention. For example, sudden changes in blood test results, such as elevated white cell counts or unexpected shifts in glucose levels, can trigger alerts for possible infections or changes in chronic conditions like diabetes.

The engine can employ a number of techniques. For example, the engine artificial intelligence (AI) techniques to enhance its diagnostic capabilities. Machine learning algorithms can analyze vast datasets to find subtle patterns that may not be evident to human observers. For instance, AI can predict the onset of conditions such as heart disease by analyzing trends over time in heart rate variability, blood pressure readings, and other cardiovascular markers. Deep learning can also be used to interpret complex imaging studies, such as MRIs or CT scans, to identify early signs of conditions like cancer or neurological disorders.

In addition or alternatively, various embodiments use AI or non-AI predictive analytics (e.g., using predictive models developed over the years) to forecast the likelihood of developing certain conditions, including use of a combination of genetic information, lifestyle data, and environmental factors. These models can assess risk levels for diseases such as Alzheimer's or certain cancers, prompting early preventive measures or more focused monitoring.

Next, step 2006 prompts the clinical knowledge database to provide resources (e.g., educational materials) relating to diagnosing and treating the determined potential patient conditions. FIG. 3 schematically shows an example of the UX, which displays patient data and a drop-down menu that enables a user to implement this search. For example, in response to a user request via the UX, the system can automatically locate and recommend clinical resources (e.g., in the clinical knowledge/resource storage) related to specific conditions through the integration of advanced search algorithms and access to comprehensive medical databases. After the condition is identified, the system searches through an extensive database that includes academic journals, such as N ature, medical research libraries, commercial resources, and other authoritative sources of clinical data.

The search mechanism may be enhanced by machine learning models that learn from interactions and improve over time. These models can prioritize and suggest the most relevant articles based on the context of the query and the user's profile, which may include the user's specialty and previous search behavior. For instance, if a cardiologist queries information about a rare heart condition, the platform can filter and present high-impact articles, recent studies, and reviews specifically focusing on cardiology from reputed sources. The platform might also connect to continuously updated databases, ensuring that the information provided is not only relevant but also the most current.

Additionally, the platform can facilitate deeper integration by linking with digital libraries and subscription-based services to which institutions typically have access. By doing so, it can provide seamless access to full articles directly through the platform's interface, bypassing the need for multiple logins and searches across different databases. This streamlined access to information saves valuable time for healthcare providers and enhances their ability to apply the latest research findings and clinical practices to patient care efficiently.

The method concludes at step 2008, which displays links to resources or the resources themselves on the UX. This also may display patient information on the same display.

By recognizing possible patient conditions and prompting high-quality educational resources, the system helps the clinician have rapid and simplified access the right information at the right time. As noted, illustrative embodiments are not considered a diagnostic tool, as it does not provide a specific, clinical diagnosis to the patient. Instead, it facilitates the clinician's ability to make an informed diagnosis with the right resources.

Illustrative embodiments preferably integrate with a larger system 100, as described herein. Of course, those figures are examples and not intended to limit some embodiments.

FIG. 19B schematically shows a clinical patient environment in accordance with illustrative embodiments of the invention. The environment includes sensors 402 (also referred to as monitors 402) for providing patient data to health providers, such as physicians, nurses, or other medical care providers. To that end, a patient 401 may be coupled to one or more physiological sensors 402 or bedside monitors 402 that may monitor various physiological parameters of the patient. It should be noted that a patient 101 may be a human, or not human (a non-human being, e.g., a dog).

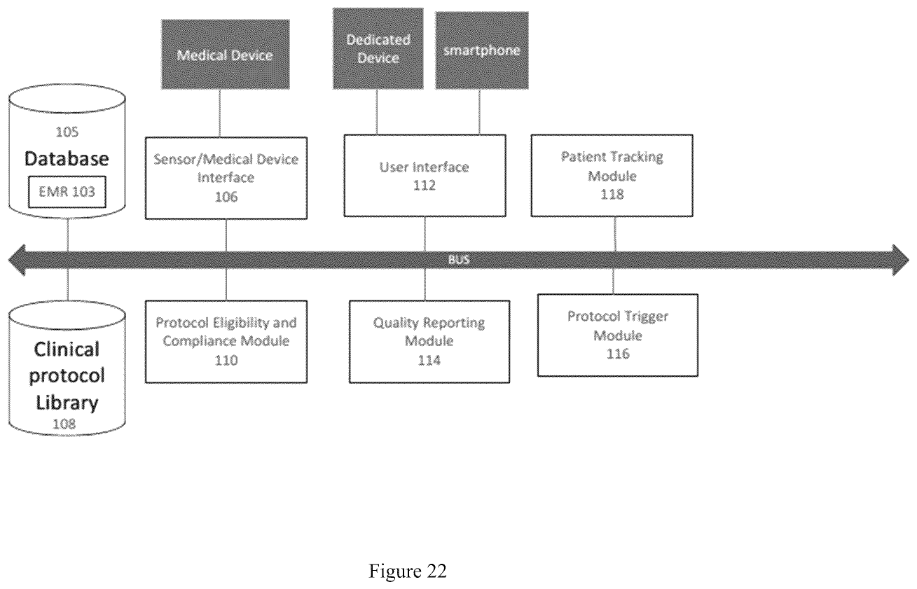

The sensors 102 may include, but are not limited to, a blood oximeter, a blood pressure measurement device, a pulse measurement device, a glucose measuring device, one or more analyte measuring devices, an electrocardiogram recording device, amongst others. In addition, the patient 401 may be administered routine exams and tests and the data may be stored in an electronic medical record (EMR) 103 (shown in FIG. 22). The electronic hemoglobin, arterial and venous oxygen content, lactic acid, weight, age, sex, ICD-10 code, capillary refill time, subjective clinician observations, patient self-evaluations, prescribed medications, medications regiments, genetics, test results, allergies, etc.

In addition, the patient 101 may be coupled to one or more treatment devices 104 that are configured to administer treatments to the patient. In some embodiments, one or more treatment devices 104 may be controlled by a system 100 as disclosed herein, for example in response to output defining a patient state or medical condition from a trajectory interpreter module. In various embodiments, the treatment devices 404 may include extracorporeal membrane oxygenator, mechanical ventilator, medication infusion pumps, implantable ventricular assist devices, etc.

Some embodiments augment the educational functionality to provide real-time automatic determination of whether the patient 101 is eligible for a course of action of the medical protocol (referred to generally as being eligible for the protocol) based at least partially on data acquired directly from the sensors and peripheral devices. The real-time determination is advantageous over prior art methods that require the medical practitioner to review disparate patient data reports and analyze the data to determine whether the patient 101 is eligible for the protocol. Additionally, the medical practitioner generally checks patient eligibility sporadically, leading to sub-optimal clinical outcomes in cases where optimal medical treatment for the patient 101 requires early initiation of the protocol. In a similar manner, sporadic eligibility checks fail to account for the dynamic concordance of the patient's eligibility because the measurements are taken at a static moment in time.

The system 100 may track how long a patient variable has been in compliance with a set eligibility criteria, advantageously improving medical protocol technology by enabling new protocols that have a longitudinal (e.g., sustained time basis) criteria, as opposed to protocols that use the most-recent static data points. Accordingly, illustrative embodiments track dynamic concordance, or the rate at which patient physiological data is consistent with the requirements of a protocol after the protocol is initiated. This advantageously allows medical practitioners to visualize the patient data as a percentage compliance per time duration (e.g., 60% dynamic concordance in the 3 hours since protocol began). Furthermore, tracking eligibility time advantageously allows medical practitioners to prioritize patients who have been eligible for a long time, thus mitigating any risk emanating from the fact that they have not been getting the prescribed protocol treatment for prolonged duration.

The dynamic concordance calculation based on longitudinal measurements of patient variables also supports using the metric as an actionable therapeutic target and consequently administering treatment targeted at increasing the concordance rate of the patient. Furthermore, the inventor has preliminary data indicating that patients achieving a desired dynamic concordance rate may experience better outcomes relative to patients that have lower concordance rate.

By providing continuous real-time assessment of whether the patient trajectory is progressing according to a protocol specified pathway, the dynamic concordance affords more focused and timely intervention to keep the patient within the most advantageous course, which is a significant improvement relative to what is possible with current technology. The longer time the patient spends within the desired trajectory as measured by the concordance rate the higher is the likelihood that the patient will not experience complications and achieve the optimal possible clinical outcome. Conversely, the less time the patient spends within the desired trajectory as measured by the concordance rate the higher the likelihood that the patient will experience complications and not achieve the optimal clinical outcome.

To that end, a patient protocol eligibility and compliance system, generally referred to herein as system 100, may be configured to receive patient related information, including real-time information from the sensors 402, EMR patient information from the electronic medical record 103, information from the treatment devices 104, such as ventilatory settings, infusion rates, types of medications, and other patient related information, which may include the patient's medical history, previous treatment plans, results from previous and present lab work, allergy information, predispositions to various conditions, and any other information that may be deemed relevant to make.

The system 100 provides real-time protocol eligibility updates. Additionally, illustrative embodiments may determine the extent of time that the patient 101 is eligible for a protocol. Accordingly, medical practitioners may have quantifiable standards by which medical practitioner performance is judged (e.g., time from eligibility to protocol initiation, protocol compliance rate, etc.).

FIG. 22 schematically shows details of the system 100 in accordance with illustrative embodiments of the invention. The system 100 includes a sensor/medical device interface 106 configured to communicate with the one or more sensors 102 and/or one or more medical devices 104. For convenience, illustrative embodiments may refer to receiving data from one or more sensors and/or one or more medical devices generally as receiving data from the sensors 102. However, it should be understood that discussion of receiving data from the sensors 102 is intended to also include receiving data from the medical devices 104.

To reiterate, the interface 106 receives/streams real-time patient data from the sensors 102. The interface 106 may receive data from a variety of sensors 102, such as blood oximeter, a blood pressure measurement device, a pulse measurement device, a glucose measuring device, one or more analyte measuring devices, an electrocardiogram recording device, amongst others. In some embodiments, the interface 106 simultaneously communicates with a plurality of sensors 102 and/or medical devices. Accordingly, the interface 106 may aggregate and/or compile the various received patient data.

The system 100 includes a database 105 having access to the patient EMR 103, as described previously. The database 105 may also communicate with the sensor interface 106 to store the real-time data as it is received via the interface 106. The system 100 also includes a medical protocol library 108 containing information relating to a plurality of medical protocols. As an example, the medical protocol may include an extubation readiness trial protocol, ventilation vasoactive weaning protocol, sepsis management protocol, enhanced recovery after surgery (ERAS) protocols, and/or other hospital protocols. The information in the library 108 includes eligibility rules and compliance rules for each of the protocols, which are discussed in more detail further below. The library 108 may also include specific subscription rules and/or notification rules for each protocol, which are discussed in more detail further below.

A protocol eligibility and compliance module 110 is configured to determine patient eligibility for at least one of the medical protocols as a function of the received patient data. Additionally, the eligibility and compliance module 110 may access historic data and/or the EMR 103 from the database 105 to determine patient eligibility. To that end, the eligibility and compliance module 110 may communicate with, among other things, the interface 106, the database 105, and the library 108.

Generally, patient eligibility depends on one or more measurements of patient data meeting a threshold status according to the requirements of the protocol stored in the library 108 (e.g., a certain FiO2 and/or BPM measurement). When the patient 101 is eligible for a protocol, the eligibility module 110 triggers an indication that the patient 101 is eligible. In some embodiments, the eligibility module 110 sounds an alert and/or communicates with a user interface 112 to visually indicate the patient is eligible. In some embodiments, the user interface also communicates the amount of time the patient has been eligible. Thus, illustrative embodiments advantageously provide real-time updates as to patient eligibility status. The real-time data collection and eligibility updates contrast to prior art methods that rely on medical practitioners checking, collecting, aggregating, and analyzing a plurality of static patient parameters to determine protocol eligibility.

The determination of eligibility is based on a collection of measurement thresholds, events, and/or other binary variables. For example, eligibility rules for a vasoactive weaning protocol may include that the patients are under cardiac service (determined by the hospital ADT stream or EMR), are on medication drip (another event), have IDO2 index less than a threshold value, and have blood pressure greater than another threshold value.

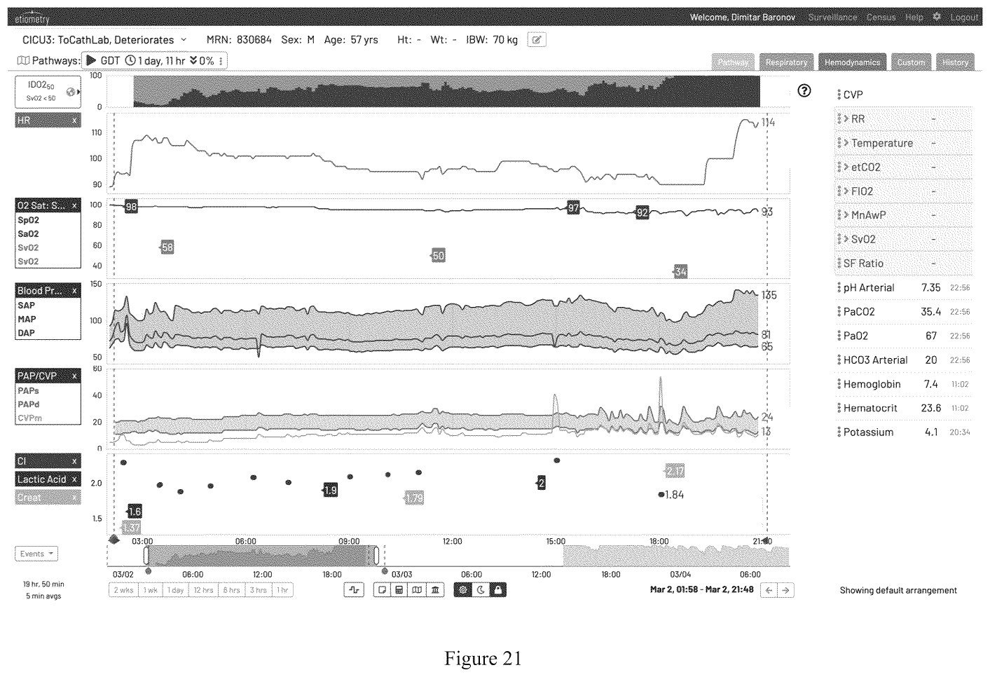

The system 100 may include a quality reporting module 114 that tracks the time elapsed from patient eligibility until a user acknowledges patient eligibility. FIG. 21 schematically shows a popup notification through which the user may acknowledge patient eligibility (e.g., on a touch screen display via user interface 112) in accordance with illustrative embodiments of the invention. Alternatively, or additionally, the quality reporting module 114 may track the time elapsed from patient eligibility until a medical practitioner begins the course of action associated with the eligible protocol for the patient 101. By tracking this information, medical practitioner performance may be objectively measured (e.g., which practitioners are the fastest responders and what are the impacts of fast response time on clinical outcomes).

The system 100 may also include a protocol trigger module 116 that instructs the eligibility and compliance module 110 to begin tracking patient 101 compliance data. The protocol trigger module 116 is configured to receive information regarding the initiation of a course of action in the protocol 120. In various embodiments, the medical practitioner may initiate the course of action based on the feedback from the eligibility module 110 and inform the protocol trigger module 116 (e.g., through the user interface) of the course of action initiation. In some embodiments, the protocol trigger module 116 is configured to automatically initiate the course of action by communicating with a treatment device 104 (e.g., after a medical practitioner approves the initiation via the user interface). In other embodiments, the protocol trigger module 116 can automatically detect that the practitioner has initiated the course of action and initiate tracking concordance data. For example, in the ERT protocol, when the system 100 detects that the ventilator mode has been changed, and this is used to detect the start of an ERT protocol and triggers dynamic concordance tracking for the protocol.

After the course of action for the protocol is initiated, the concordance module 110 may continue to communicate with the same, different, or additional sensors 102 to determine patient 101 compliance with the protocol. The compliance module 110 may determine the compliance of the patient 101 with the medical protocol as a function of newly received patient data and the compliance rules in the library 108. Compliance is generally determined as a calculation of whether the patient data meets acceptable levels set by the library 108 for the particular protocol (e.g., the patient variable is compared to the level or range required by the library 108). Additionally, the compliance module 110 may visually indicate (e.g., via the user interface 112) the real-time patient dynamic concordance status. FIG. 19B schematically shows eligibility rules 122 and compliance rules 124 for an example of an extubation readiness test protocol in accordance with illustrative embodiments. It should be understood that the tracked parameters for eligibility may differ from the tracked parameters for compliance, as shown in FIG. 19B. The concordance module 110 may send the dynamic concordance rate to the user interface 112, which displays the dynamic concordance rate. Accordingly, the attention of the medical practitioner may be drawn to the patient in need of action.

The quality reporting module 114 may track a dynamic concordance rate as a percentage of time over which the patient 101 is compliant with the medical protocol and/or the patient eligibility time. Additionally, the quality reporting module 114 may retrospectively look at patient concordance rate and/or eligibility time to see how well a particular patient and/or patients of a particular medical practitioner have performed relative to a patient population. Additionally, the reporting module 114 may be configured to display the received patient data and to mark non-compliant portions of the received data. Thus, the quality reporting module 114 allows medical facilities and/or staff to evaluate how well patients were managed retrospectively. For example, if eligibility times are very high, medical practitioners become aware of it. This assists with hospital policy formation and also with ensuring prompt action by the medical staff.

The system of FIG. 22 may include a risk-based monitoring engine 1000 (also referred to as “risk engine 1000”) configured to receive data from bedside monitors 102, electronic medical records 103, treatment devices 104, and any other information that may be deemed relevant to make an informed assessment regarding the patient's clinical risks, and any combination thereof of the preceding elements.

In illustrative embodiments, the risk engine 1000 may include a physiology observer module 119 that utilizes multiple measurements to estimate probability density functions (PDF) of internal state variables (ISVs) that describe the components of the physiology relevant to the patient treatment and condition in accordance with a predefined physiology model. The ISVs may be directly observable with noise (as a non-limiting example, heart rate is a directly observable ISV), hidden (as a non-limiting example, oxygen delivery (DO2) defined as the flow of blood saturated oxygen through the aorta cannot be directly measured and is thus hidden), or measured intermittently (as a non-limiting example, hemoglobin concentration as measured from Complete Blood Count tests is an intermittently observable ISV).

In some embodiments, when the physiology observer module 119 evaluates a set of ISVs at a given time step (e.g., tk; tk+1; generally tk+n), the system 100 may not have a complete set of ISV measurements contemporaneous with that given time step. For example, the system 100 may have measurements for that given time step for some internal state variables, but may not have measurements for that given time step for some other internal state variables (e.g., a contemporaneous measurement for an intermittent ISV may not be available for the given time step). Consequently, that intermittent ISV is, for purposes of evaluating ISVs at the given time step, a hidden ISV. However, evaluation of the set of ISVs by the physiology observer module 119 (as described herein) is nevertheless possible according to embodiments described herein because the predicted PDFs of ISVs 211 carry in them the influence of past measurements of that intermittent ISV, and consequently those predicted PDFs of ISVs 211 are, in illustrative embodiments, sufficient input for the physiology observer module 119.

In one embodiment, instead of assuming that all variables can be estimated deterministically without error, the physiology observer module 119 of the present disclosure provides probability density functions as an output. Additional details related to the physiology observer module 119 are provided herein.

The clinical trajectory interpreter module 123 may be configured, for example, with multiple possible patient states, and may determine which of those patient states are probable and with what probability, given the estimated probability density functions of the internal state variables. Examples of particular patient states include, but are not limited to, hypotension with sinus tachycardia, hypoxia with myocardial depression, compensated circulatory shock, cardiac arrest, hemorrhage, amongst others. In addition, these patient states may be specific to a particular medical condition, and the bounds of each of the patient states may be defined by threshold values of various physiological variables and data. In various embodiments, the clinical trajectory interpreter module 123 may determine the patient conditions under which a patient may be categorized using any of information gathered from reference materials, information provided by health care providers, other sources of information.

The reference materials may be stored in the database 105 or other storage device that is accessible to the risk-based monitoring application 1020 via network interface 113, for example. These reference materials may include material synthesized from reference books, medical literature, surveys of experts, physician provided information, and any other material that may be used as a reference for providing medical care to patients. In some embodiments, the clinical trajectory interpreter module 123 may first identify a patient population that is similar to the subject patient being monitored. By doing so, the clinical trajectory interpreter module 123 may be able to use relevant historical data based on the identified patient population to help determine the possible patient states.

The clinical trajectory interpreter module 123 is capable of also determining the probable patient states under which the patient can be currently categorized, given the estimated probability density functions of the internal state variables, as provided by physiology observer module 122. In this way, each of the possible patient states is assigned a probability value from 0 to 1. The combination of patient states and their probabilities is defined as the clinical risk to the patient.

Each of the above-described components is operatively connected by any conventional interconnect mechanism. FIG. 22 simply shows a bus communicating each of the components. Those skilled in the art should understand that this generalized representation can be modified to include other conventional direct or indirect connections. Accordingly, discussion of a bus is not intended to limit various embodiments.

Indeed, it should be noted that FIG. 22 only schematically shows each of these components. Those skilled in the art should understand that each of these components can be implemented in a variety of conventional manners, such as by using hardware, software, or a combination of hardware and software, across one or more other functional components. For example, the quality reporting module 114 may be implemented using a plurality of microprocessors executing firmware. As another example, the protocol eligibility and compliance module 110 may be implemented using one or more application specific integrated circuits (i.e., “ASICs”) and related software, or a combination of ASICs, discrete electronic components (e.g., transistors), and microprocessors. Accordingly, the representation of the components in a single box of FIG. 22 is for simplicity purposes only.

In some embodiments, components of the system may be separated into different components. For example, the protocol eligibility and compliance module may be split into two different modules, e.g., a protocol eligibility module and a protocol compliance module. Additionally, various components, such as the sensor/medical device interface 106 of FIG. 22, may be distributed across a plurality of different machines—not necessarily within the same housing or chassis.

Additionally, in some embodiments, components shown as separate (such as the eligibility and compliance module 110 and the quality reporting module 114 in FIG. 22) may be replaced by a single component. Illustrative embodiments may include additional modules not explicitly shown here. Furthermore, certain components and sub-components in FIG. 22 are optional. For example, some embodiments may not include the protocol trigger module 116.

It should be reiterated that the representation of FIG. 22 is a significantly simplified representation of the system 100. Those skilled in the art should understand that such a device may have other physical and functional components, such as central processing units, other packet processing modules, and short-term memory. Indeed, the system 100, in various embodiments, includes one or more of the following: a processor, a memory coupled to the processor, and a network interface configured to enable the system to communicate with other devices over a network. In addition, the system may include a risk-based monitoring application 1020 that may include computer-executable instructions, which when executed by the processor 111, cause the system to be able to afford risk-based monitoring of the patients, such as the patient 101. Accordingly, this discussion is not intended to suggest that FIG. 22 represents all of the elements of the system 100.

This and related embodiments collaborating with the knowledge platform may be similar to those in U.S. Pat. No. 11,763,947, co-owned by the assignee of this provisional patent application, the disclosure of which is incorporated herein, in its entirety, by reference.

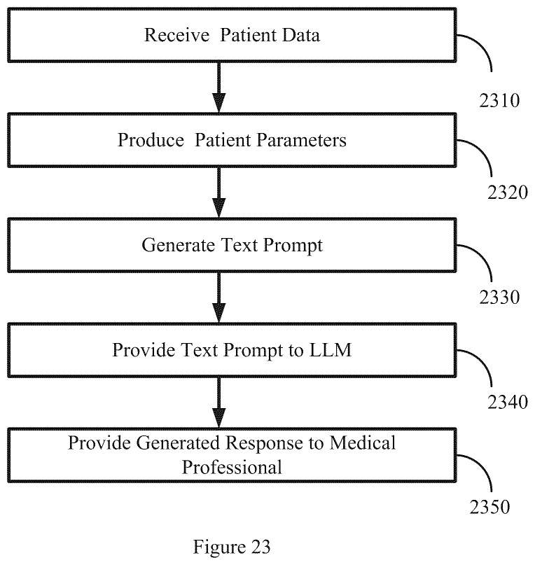

FIG. 23 is a flowchart of an embodiment of a method.

Step 2310 includes receiving patient data for a specific patient. Patient data may be generated by one or more sensors 102, one or more treatment devices 104, and/or one or more embodiments of a system 100.

Step 2320 include producing patient parameters. Patient parameters may be produce, for example, one or more embodiments of a system 100.

Step 2330 includes generating a text prompt from the patient parameters.

Step 2340 includes providing the text prompt to a large language model (“LLM”).

Step 2350 includes receiving a response from the LLM, and providing that response to a medical professional treating and/or responsible for treating the patient.

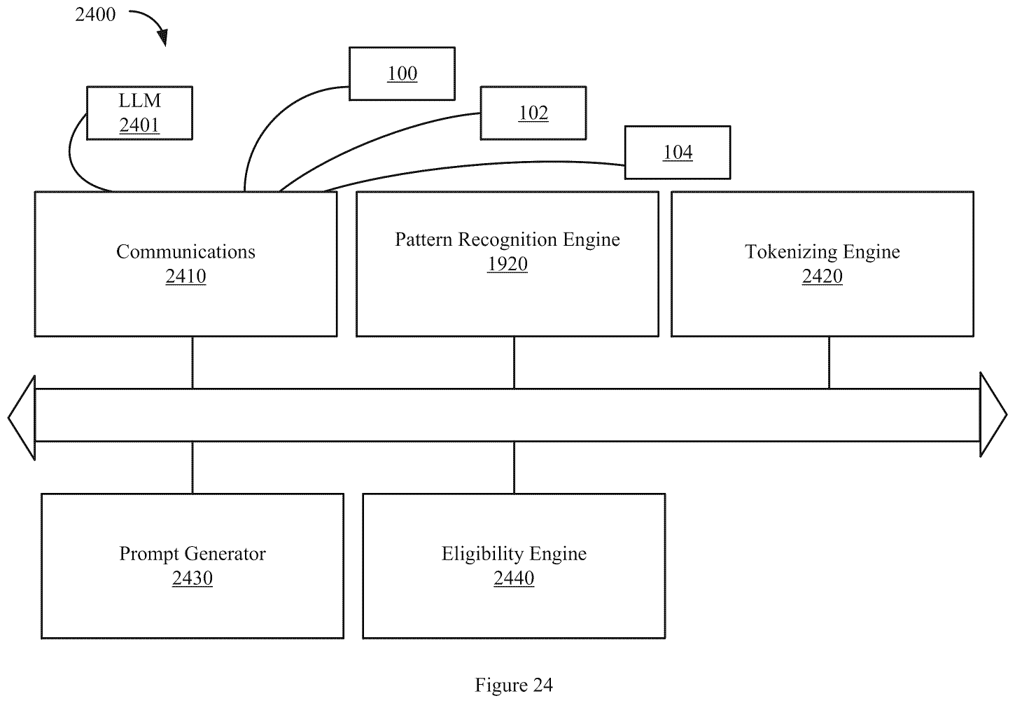

FIG. 24 schematically illustrates a system 2400.

The system 2400 includes a communications interface configured in data communication with one or more of an LLM 2401, a system 100 according to the embodiment of such systems 100 described herein, one or more sensors 102, and one or more monitors or treatment devices 104 coupled to a patient.

System 2400 includes a pattern recognition engine 1920 as described herein.

System 2400 includes a tokenization engine 2420 configured to perform tokenization processes, as described herein.

System 2400 includes a prompt generator engine 2430 configured to generate prompts, as described herein.

System 2400 includes an eligibility engine 2440 configured to determine a patient's eligibility for a specific protocol, and/or a patient's concordance with the specific protocol, as described herein.

Various embodiments may be characterized by the potential claims listed in the paragraphs following this paragraph (and before the actual claims provided at the end of this application). These potential claims form a part of the written description of this application. Accordingly, subject matter of the following potential claims may be presented as actual claims in later proceedings involving this application or any application claiming priority based on this application. Inclusion of such potential claims should not be construed to mean that the actual claims do not cover the subject matter of the potential claims. Thus, a decision to not present these potential claims in later proceedings should not be construed as a donation of the subject matter to the public.

Without limitation, potential subject matter that may be claimed (prefaced with the letter “P” so as to avoid confusion with the actual claims presented below) includes:

P1. A computer-implemented method comprising:
receiving, at a computer system, patient data describing patient physiology, said patient data including at least data quantifying a set of patent state variables from a set of sensors coupled to the patient;
producing, from the patient data, a set of patient parameters for the patient, said patient parameters comprising:
(1) a probability that the patient is in a specific patient state, and
tokenizing the patient parameters to produce a set of text descriptions; and
producing, from the set of text descriptions, a structured text prompt configured as input to a large language model, said structured text prompt configured to cause the large language model to generate at least one of:
(a) a set of proposed diagnoses of the patient, each proposed diagnosis including a quantitative probabilistic assessment;
(b) a physiological interpretation of patterns observed within the patient parameters;
(c) a listing of relevant clinical considerations;
(d) a listing of relevant clinical caveats to the set of proposed diagnoses;
(e) a suggestion of an additional assessment to be made for the patient;
(f) a suggestion for additional monitoring of the patient;
(g) a listing of clinical guidelines relevant to a specific patient state or diagnosis of the patient; and
(h) a listing of literature relevant to a specific patient state or diagnosis of the patient;
(i) an indication whether the patient is compliant with a particular protocol or is not compliant with a particular protocol.

P2. The method of P1 wherein producing, from the patient data, a set of patient parameters for the patient comprises producing (2) a quantitative indicator of patient eligibility for a treatment protocol.

P3. The innovation of P2 wherein, the structured text prompt configured to cause the large language model to generate (j) an indication whether the patient is compliant with a particular protocol or is not compliant with a particular protocol.

P4. The method of any of P1-P3 wherein tokenizing, by the computer system, the patient parameters to produce a set of text descriptions comprises:
correlating the patient parameters to a pre-existing form text string; and
modifying the pre-existing form text string to add the patient parameters.

P5. The method of any of P1-P4 wherein tokenizing, by the computer system, the patient parameters to produce a set of text descriptions comprises:
providing the patient parameters to a neural network trained to recognize a specific pattern within patient parameters; and
receiving an output from the neural network which output correlates the patient parameters to a pre-existing form text string which pre-existing form text string correlated to the specific pattern; and
modifying the pre-existing form text string to add the patient parameters.

P6. The method of any of P1-P5 wherein:
the patient parameters comprise a plurality of patient parameters spanning a length of time;
the set of text descriptions comprises text describing a trend over the length of time, the trend represented by the plurality of patient parameters spanning a length of time.

P7. The method of P6 wherein:
the patient parameters define an evolution of the patient state over time.

8. The method of P2 wherein set of text descriptions comprise a description of the patient's eligibility for the specified treatment protocol.

P9. The method of P2 wherein set of text descriptions comprise a description of the patient's concordance with the specified treatment protocol.

P10. The method innovation of any of P1-P9 wherein the set of text descriptions comprises a request to identify literature relating to a specific patient state.

P11. A computer-implemented system comprising:
a communications interface configured to receive patient data describing patient physiology, said patient data including at least data quantifying a set of patent state variables from a set of sensors coupled to the patient;
a pattern recognition engine configured to produce, from the patient data, a set of patient parameters for the patient, said patient parameters comprising:
(1) a probability that the patient is in a specific patient state;
a tokenizing engine configured to produce, from the patient parameters, a set of text descriptions; and
a prompt generator configured to produce, from the set of text descriptions, a structured text prompt configured as input to a large language model, said structured text prompt configured to cause the large language model to generate at least one of:
(a) a set of proposed diagnoses of the patient, each proposed diagnosis including a quantitative probabilistic assessment;
(b) a physiological interpretation of patterns observed within the patient parameters;
(c) a listing of relevant clinical considerations;
(d) a listing of relevant clinical caveats to the set of proposed diagnoses;
(e) a suggestion of an additional assessment to be made for the patient;
(f) a suggestion for additional monitoring of the patient;
(g) a listing of clinical guidelines relevant to a specific patient state or diagnosis of the patient; and
(h) a listing of literature relevant to a specific patient state or diagnosis of the patient;
(i) an indication whether the patient is compliant with a particular protocol.

P12. The system of claim P11, the patient parameters further comprising: (2) a quantitative indicator of patient eligibility for a specified treatment protocol.

P13. The system of P12 wherein the structured text prompt configured to cause the large language model to generate (j) an indication whether the patient is compliant with a specified protocol.

P14. The system of any of P11-P13 wherein the tokenizing engine is configured to produce the set of text descriptions by:
correlating the patient parameters to a pre-existing form text string; and
modifying the pre-existing form text string to add the patient parameters.

P15. The system of any of P11-P14 further comprising:
a neural network trained to recognize a specific pattern within patient parameters; and wherein the tokenizing engine is configured to:
receive an output from the neural network which output correlates the patient parameters to a pre-existing form text string which pre-existing form text string correlated to the specific pattern; and
modify the pre-existing form text string to add the patient parameters.

P16. A non-transitory computer-readable medium having computer executable code thereon, the computer executable code, when executed by a computer system, causing the computer system to perform a method, the code comprising:
code for receiving, at the computer system, patient data describing patient physiology, said patient data including at least data quantifying a set of patent state variables from a set of sensors coupled to the patient;
code for producing, by the computer system from the patient data, a set of patient parameters for the patient, said patient parameters comprising:
(1) a probability that the patient is in a specific patient state;
code for tokenizing, by the computer system, the patient parameters to produce a set of text descriptions; and
code for producing, by the computer system, from the set of text descriptions, a structured text prompt configured as input to a large language model, said structured text prompt configured to cause the large language model to generate at least one of:
(a) a set of proposed diagnoses of the patient, each proposed diagnosis including a quantitative probabilistic assessment;
(b) a physiological interpretation of patterns observed within the patient parameters;
(c) a listing of relevant clinical considerations;
(d) a listing of relevant clinical caveats to the set of proposed diagnoses;
(e) a suggestion of an additional assessment to be made for the patient;
(f) a suggestion for additional monitoring of the patient;
(g) a listing of clinical guidelines relevant to a specific patient state or diagnosis of the patient; and
(h) a listing of literature relevant to a specific patient state or diagnosis of the patient;
(i) an indication whether the patient is compliant with a particular protocol.

P17. The non-transitory computer-readable medium of P16, wherein producing, from the patient data, a set of patient parameters for the patient comprises producing (2) a quantitative indicator of patient eligibility for a specified treatment protocol.

P18. The non-transitory computer-readable medium of any of P16-P17 wherein tokenizing the patient parameters to produce a set of text descriptions comprises:
correlating the patient parameters to a pre-existing form text string; and
modifying the pre-existing form text string to add the patient parameters.

P19. The non-transitory computer-readable medium of any of P16-P18, wherein tokenizing the patient parameters to produce a set of text descriptions comprises:
providing the patient parameters to a neural network trained to recognize a specific pattern within patient parameters; and
receiving an output from the neural network which output correlates the patient parameters to a pre-existing form text string which pre-existing form text string correlated to the specific pattern; and
modifying the pre-existing form text string to add the patient parameters.

P20. The non-transitory computer-readable medium of any of P16-P19, wherein:
the patient parameters comprise a plurality of patient parameters spanning a length of time;
the set of text descriptions comprises text describing a trend over the length of time, the trend represented by the plurality of patient parameters spanning a length of time.

P31. A computer-implemented system comprising a computer configured to perform a method comprising any of P1-P10.

P41. A non-transitory computer-readable medium having computer executable code thereon, the computer executable code, when executed by a computer system, causing the computer system to perform a method comprising any of P1-P10.

P51: A clinical educational method comprising:
receiving patient data;
using the patient data to identify one or more possible conditions;
directing a user to access a resource as a function of the one or more possible conditions.

P52. The method of P511 wherein the patient data comprises current clinical information about the patient and/or stored clinical information about the patient.

P53. The method of any one or more of P51-P52 wherein the patient data includes one or more of age, gender, demographics, treatment history, family medical history, medicines, allergies, diagnostic test results, and immunization records.

P54. The method of any one or more of P51-P53 wherein the resource comprises a database having clinical information.

P55. The method of any one or more of P51-P54 further comprising displaying the resource on a display device in a graphical user interface.

P56. The method of any one or more of P51-P55 wherein directing a user comprises directing a user to access the resource across the Internet.

P57. The method of any one or more of P51-P56 further comprising displaying a graphical user interface of patient clinical information, the graphical user interface having indicia for selecting the resource.

P58. The method of any one or more of P51-P57 further comprising not delivering a clinical diagnosis.

P59. A system implementing any or more of the above innovations of P51-P58.

P60. A computer program process executing on a tangible medium having program readable code configured to implement the method of any one or more of the methods of P51-P58.

Various embodiments of this disclosure may be implemented at least in part in any conventional computer programming language. For example, some embodiments may be implemented in a procedural programming language (e.g., “C”), or in an object-oriented programming language (e.g., “C++”), or in Python, R, Java, LISP or Prolog. Other embodiments of this disclosure may be implemented as preprogrammed hardware elements (e.g., application specific integrated circuits, FPGAs, and digital signal processors), or other related components.

In an alternative embodiment, the disclosed apparatus and methods may be implemented as a computer program product for use with a computer system. Such implementation may include a series of computer instructions fixed either on a tangible medium, such as a non-transitory computer readable medium (e.g., a diskette, CD-ROM, ROM, FLASH memory, or fixed disk). The series of computer instructions can embody all or part of the functionality previously described herein with respect to the system.

Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies.

Among other ways, such a computer program product may be distributed as a removable medium with accompanying printed or electronic documentation (e.g., shrink wrapped software), preloaded with a computer system (e.g., on system ROM or fixed disk), or distributed from a server or electronic bulletin board over the network (e.g., the Internet or World W ide Web). Of course, some embodiments of this disclosure may be implemented as a combination of both software (e.g., a computer program product) and hardware. Still other embodiments of this disclosure are implemented as entirely hardware, or entirely software.

Computer program logic implementing all or part of the functionality previously described herein may be executed at different times on a single processor (e.g., concurrently) or may be executed at the same or different times on multiple processors and may run under a single operating system process/thread or under different operating system processes/threads. Thus, the term “computer process” refers generally to the execution of a set of computer program instructions regardless of whether different computer processes are executed on the same or different processors and regardless of whether different computer processes run under the same operating system process/thread or different operating system processes/threads.

The embodiments described above are intended to be merely exemplary; numerous variations and modifications will be apparent to those skilled in the art. All such variations and modifications are intended to be within the scope of the present disclosure as defined in any appended claims.

What is claimed is:

1. A computer-implemented method comprising:
receiving, at a computer system, patient data describing patient physiology, said patient data including at least data quantifying a set of patent state variables from a set of sensors coupled to the patient;
producing, by the computer system from the patient data, a set of patient parameters for the patient, said patient parameters comprising:
(1) a probability that the patient is in a specific patient state;
tokenizing, by the computer system, the patient parameters to produce a set of text descriptions; and
producing, by the computer system, from the set of text descriptions, a structured text prompt configured as input to a large language model, said structured text prompt configured to cause the large language model to generate at least one of:
(a) a set of proposed diagnoses of the patient, each proposed diagnosis including a quantitative probabilistic assessment;
(b) a physiological interpretation of patterns observed within the patient parameters;
(c) a listing of relevant clinical considerations;
(d) a listing of relevant clinical caveats to the set of proposed diagnoses;
(e) a suggestion of an additional assessment to be made for the patient;
(f) a suggestion for additional monitoring of the patient;
(g) a listing of clinical guidelines relevant to a specific patient state or diagnosis of the patient;
(h) a listing of literature relevant to a specific patient state or diagnosis of the patient;
(i) an indication whether the patient is compliant with a particular protocol.

2. The method of claim 1 wherein producing, from the patient data, the set of patient parameters for the patient comprises producing (2) a quantitative indicator of patient eligibility for a specified treatment protocol.

3. The method of claim 2 wherein the structured text prompt configured to cause the large language model to generate (j) an indication whether the patient is compliant with a specified treatment protocol.

4. The method of claim 1 wherein tokenizing, by the computer system, the patient parameters to produce a set of text descriptions comprises:
correlating the patient parameters to a pre-existing form text string; and
modifying the pre-existing form text string to add the patient parameters.

5. The method of claim 1 wherein tokenizing, by the computer system, the patient parameters to produce a set of text descriptions comprises:
providing the patient parameters to a neural network trained to recognize a specific pattern within patient parameters; and
receiving an output from the neural network which output correlates the patient parameters to a pre-existing form text string which pre-existing form text string correlated to the specific pattern; and
modifying the pre-existing form text string to add the patient parameters.

6. The method of claim 1 wherein:
the patient parameters comprise a plurality of patient parameters spanning a length of time;
the set of text descriptions comprises text describing a trend over the length of time, the trend represented by the plurality of patient parameters spanning a length of time.

7. The method of claim 6 wherein:
the patient parameters define an evolution of the patient state over time.

8. The method of claim 2 wherein set of text descriptions comprise a description of the patient's eligibility for the specified treatment protocol.

9. The method of claim 2 wherein set of text descriptions comprise a description of the patient's concordance with the specified treatment protocol.

10. The method of claim 1 wherein set of text descriptions comprises a request to identify literature relating to a specific patient state.

11. A computer-implemented system comprising:
a communications interface configured to receive patient data describing patient physiology, said patient data including at least data quantifying a set of patent state variables from a set of sensors coupled to the patient;
a pattern recognition engine configured to produce, from the patient data, a set of patient parameters for the patient, said patient parameters comprising:
(1) a probability that the patient is in a specific patient state;
a tokenizing engine configured to produce, from the patient parameters, a set of text descriptions; and
a prompt generator configured to produce, from the set of text descriptions, a structured text prompt configured as input to a large language model, said structured text prompt configured to cause the large language model to generate at least one of:
(a) a set of proposed diagnoses of the patient, each proposed diagnosis including a quantitative probabilistic assessment;
(b) a physiological interpretation of patterns observed within the patient parameters;
(c) a listing of relevant clinical considerations;
(d) a listing of relevant clinical caveats to the set of proposed diagnoses;
(e) a suggestion of an additional assessment to be made for the patient;
(f) a suggestion for additional monitoring of the patient;
(g) a listing of clinical guidelines relevant to a specific patient state or diagnosis of the patient; and
(h) a listing of literature relevant to a specific patient state or diagnosis of the patient;
(i) an indication whether the patient is compliant with a particular protocol.

12. The system of claim 11, the patient parameters further comprising: (2) a quantitative indicator of patient eligibility for a specified treatment protocol.

13. The system of claim 12 wherein the structured text prompt configured to cause the large language model to generate (j) an indication whether the patient is compliant with a specified protocol.

14. The system of claim 11 wherein the tokenizing engine is configured to produce the set of text descriptions by:
correlating the patient parameters to a pre-existing form text string; and
modifying the pre-existing form text string to add the patient parameters.

15. The system of claim 11 further comprising:
a neural network trained to recognize a specific pattern within patient parameters; and wherein the tokenizing engine is configured to:
receive an output from the neural network which output correlates the patient parameters to a pre-existing form text string which pre-existing form text string correlated to the specific pattern; and
modify the pre-existing form text string to add the patient parameters.

16. A non-transitory computer-readable medium having computer executable code thereon, the computer executable code, when executed by a computer system, causing the computer system to perform a method, the code comprising:
code for receiving, at the computer system, patient data describing patient physiology, said patient data including at least data quantifying a set of patent state variables from a set of sensors coupled to the patient;
code for producing, by the computer system from the patient data, a set of patient parameters for the patient, said patient parameters comprising:
(1) a probability that the patient is in a specific patient state;
code for tokenizing, by the computer system, the patient parameters to produce a set of text descriptions; and
code for producing, by the computer system, from the set of text descriptions, a structured text prompt configured as input to a large language model, said structured text prompt configured to cause the large language model to generate at least one of:
(a) a set of proposed diagnoses of the patient, each proposed diagnosis including a quantitative probabilistic assessment;
(b) a physiological interpretation of patterns observed within the patient parameters;
(c) a listing of relevant clinical considerations;
(d) a listing of relevant clinical caveats to the set of proposed diagnoses;
(e) a suggestion of an additional assessment to be made for the patient;
(f) a suggestion for additional monitoring of the patient;
(g) a listing of clinical guidelines relevant to a specific patient state or diagnosis of the patient; and
(h) a listing of literature relevant to a specific patient state or diagnosis of the patient;
(i) an indication whether the patient is compliant with a particular protocol.

17. The non-transitory computer-readable medium of claim 16, wherein producing, from the patient data, a set of patient parameters for the patient comprises producing (2) a quantitative indicator of patient eligibility for a specified treatment protocol.

18. The non-transitory computer-readable medium of claim 16 wherein tokenizing the patient parameters to produce a set of text descriptions comprises:
correlating the patient parameters to a pre-existing form text string; and
modifying the pre-existing form text string to add the patient parameters.

19. The non-transitory computer-readable medium of claim 16, wherein tokenizing the patient parameters to produce a set of text descriptions comprises:
providing the patient parameters to a neural network trained to recognize a specific pattern within patient parameters; and
receiving an output from the neural network which output correlates the patient parameters to a pre-existing form text string which pre-existing form text string correlated to the specific pattern; and
modifying the pre-existing form text string to add the patient parameters.

20. The non-transitory computer-readable medium of claim 16, wherein:
the patient parameters comprise a plurality of patient parameters spanning a length of time;
the set of text descriptions comprises text describing a trend over the length of time, the trend represented by the plurality of patient parameters spanning a length of time.
